(19) United States
(12) Patent Application Publication
(10) Pub. No.: US 2007/0231273 A1
(43) Pub. Date: Oct. 4, 2007

(54) Method for Decreasing Blood Glucose Levels

(76) Inventor: Jie Wu, Avondale, AZ (US)

Correspondence Address: BOOTH UDALL, PLC, 1155 W. Rio Salado Pkwy., Suite 101, Tempe, AZ, 85281, US

(21) Appl. No.: 11/278,367

(22) Filed: Mar. 31, 2006

(51) Int. Cl. A61K 31/4745; A61K 9/68; A61K 9/48; A61K 9/20

(52) U.S. Cl. 424/48; 514/283; 424/440; 424/451; 424/464; 424/449

(57) ABSTRACT

A method for decreasing blood glucose levels is disclosed. Iptakalim hydrochloride (a SUR1 subunit-dependent K_{ATP} channel blocker and a SUR2 subunit-selective K_{ATP} channel opener) is used to block pancreatic β-cell K_{ATP} channels, which depolarizes β-cells, elevates intracellular Ca²⁺ concentrations, and in turn increases insulin release. Therefore, in some implementations, iptakalim hydrochloride is an optimal treatment for type-2 diabetic patients with cardiovascular disorders.

### BACKGROUND

A. Technical Field

This document relates to a method for increasing insulin release/decreasing blood glucose levels by treatment with iptakalim hydrochloride, a cardiovascular ATP-sensitive potassium (K_{ATP}) channel opener and a pancreatic β-cell K_{ATP} channel closer.

B. Background Art

Diabetes mellitus is a group of diseases characterized by high levels of blood glucose resulting from defects in insulin production, insulin action, or both. Diabetes can be associated with serious complications and premature death. An estimated 18.2 million people in the United States—6.3% of the population—have diabetes, a serious, lifelong condition. Each year, about 1.3 million people aged 20 or older are diagnosed with diabetes. In 2002, diabetes cost the United States $132 billion (including both direct and indirect costs).

Type-2 diabetes may account for more than 90% of all diagnosed cases of diabetes. Type-2 diabetes is increasingly being diagnosed in children and adolescents. Type-2 diabetes usually begins with insulin resistance, a disorder in which cells do not use insulin properly. As the need for insulin rises, the pancreas gradually loses its ability to produce insulin.

The purpose of type-2 diabetes treatment is to lower or control circulating blood glucose levels through food management, exercise and medication. More than 50% of diagnosed type-2 diabetic patients need to take medication. Current strategies to treat diabetes include reducing insulin resistance using glitazones, supplementing insulin supplies with exogenous insulin, or increasing endogenous insulin production with sulfonylureas. Sulfonylureas constitute the leading oral antihyperglycaemic agents over the past half-century. The major target of sulfonylureas is one type of potassium ion channel, called ATP-sensitive potassium (K_{ATP}) channels, which are expressed in pancreatic β-cells.

K_{ATP} channels belong to a family of inwardly rectifying potassium channel subunits (Kir6.2 or 6.1) each coupled to a sulfonylurea (SUR) binding subunit. In pancreatic β-cells, K_{ATP} channels play a critical role in the regulation of β-cell excitation and insulin secretion. The closing of K_{ATP} channels causes β-cell depolarization, in turn activates voltage-sensitive Ca²⁺ channels and increases cytosolic Ca²⁺ concentrations, thereby leading to insulin secretion. Therefore, many K_{ATP} channel closers, including tolbutamide, glyburide, gliclazide, nateglinide, repaglinide and glibenclarimade, have been used for many years for the treatment of type-2 diabetes.

Notwithstanding, K_{ATP} channels are widely expressed in a variety of tissues including cardiovascular cells, muscle cells, pancreatic β-cells and in various brain neurons, and the diversity of tissue-specific expression of SUR subunits may determine the pharmacological properties of K_{ATP} channels Among these tissues, SUR subunits have shown different expression. For example, pancreatic β-cells express Kir6.2-SUR1, myocardial cells express Kir6.2-SUR2A, while smooth muscle cells of blood vessels express Kir6.2-SUR2B. Sulfonylureas block β-cell K_{ATP} channels, while simultaneously blocking other tissues' K_{ATP} channels, causing side effects during type-2 diabetes treatment.

The diverse expression of K_{ATP} channel subunits in different tissues causes possible side effects of oral diabetic drugs (sulfonylureas). For instance, it is believed that in the heart, K_{ATP} channels play an important role in the intrinsic mechanisms that protect cardiac muscle during hypoxia/ischemia. In arterial smooth muscle, K_{ATP} channels are also important in maintaining contractile tone, in turn controlling blood pressure and blood flow. It has been reported that in type-2 diabetic patients treated with sulfonylureas (K_{ATP} channel blockers), the major cause of death is cardiovascular diseases, which has been argued that this could, at least in part, be relevant to the side effects of sulfonylureas by blocking cardiovascular K_{ATP} channels.

Therefore, the optimal, new generation of sulfonylureas is the drug that blocks pancreatic β-cell K_{ATP} channels but exhibits little blocking effects on cardiovascular K_{ATP} channels, or even better, that opens cardiovascular K_{ATP} channels. Until now, there has been no such optimal drug to meet these purposes. Although tolbutamide (first generation of sulfonylureas) and gliclazide (second generation of sulfonylureas) were reported to produce high-affinity closure of β-cell type (Kir6.2/SUR1), but not cardiac (Kir6.2/SUR2A) or smooth muscle type (Kir6.2/SUR2B), K_{ATP} channels, they exhibit little opening effects on cardiovascular K_{ATP} channels.

The development of a new drug that closes pancreatic β-cell K_{ATP} channels but opens cardiovascular K_{ATP} channels has important clinical significances. Large amounts of evidence indicate that the opening of cardiovascular K_{ATP} channels exhibits beneficial effects on cardiovascular disorders, including the protection of the myocardial system against ischemia/hypoxia, the prevention of ventricular arrhythmias and antihypertension. All of these K_{ATP} channel-opening effects will benefit type 2-diabetic patients with accompanying cardiac and blood vessel disorders.

Thus, a considerable need exists for a compound that can selectively block pancreatic β-cell K_{ATP} channels but open cardiovascular K_{ATP} channels, which will be an optimal therapeutic strategy to treat type-2 diabetes with positive benefits for cardiac and vessel systems.

### SUMMARY

In an aspect, this document features a method of reducing blood glucose levels in a living organism comprising the step of administering a therapeutically effective dose of iptakalim hydrochloride to the living organism. Iptakalim hydrochloride not only is a cardiovascular K_{ATP} channel opener, but iptakalim hydrochloride closes pancreatic β-cell K_{ATP} channels, thereby exciting β-cells, elevating intracellular Ca²⁺ concentrations, and increasing β-cell release of insulin. Thus, iptakalim hydrochloride has utility as a treatment for reducing blood glucose in type 2-diabetic patients.

Implementations may include one or more of the following. The amount administered is between about 0.5 milligrams and about 4 milligrams per kilogram of body weight of the living organism. The therapeutically effective dose of iptakalim hydrochloride is about 3 milligrams per kilogram of body weight of the living organism. An administration route for the dose of iptakalim hydrochloride is one of topical, buccal, sublingual, transdermal, oral, rectal, ophthalmic, intravitreal, intracameral, nasal, vaginal, parenteral, subcutaneous, intramuscular, intravenous, intradermal, intratracheal, epidural, and combinations thereof. The dose of iptakalim hydrochloride is administered in a form of one of a capsule, a cachet, a pill, a tablet, a powder, a granule, a pellet, a bead, a particle, a gum, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, a foam, a food product, and combinations thereof. The method may further comprise the step of administering one of an antihyperglycaemic/antihypertensive agent, an antihyperglycaemic agent, an antihypertensive agent, and combinations thereof. The method may further comprise administering a therapeutically effective dose of a combination therapy comprising iptakalim hydrochloride and one of an antihyperglycaemic/antihypertensive agent, an antihyperglycaemic agent, an antihypertensive agent, and combinations thereof. The living organism is a mammal. The mammal is a human.

In another aspect, this document features a method of treating Type-2 diabetes comprising administering to a patient in need thereof a safe and therapeutically effective amount of a composition comprising iptakalim hydrochloride.

Implementations may include one or more of the following. The amount administered of iptakalim hydrochloride is between about 0.5 milligrams and about 4 milligrams per kilogram of body weight of the patient. The amount administered of iptakalim hydrochloride is about 3 milligrams per kilogram of body weight of the patient. An administration route for the dose of iptakalim hydrochloride is one of topical, buccal, sublingual, transdermal, oral, rectal, ophthalmic, intravitreal, intracameral, nasal, vaginal, parenteral, subcutaneous, intramuscular, intravenous, intradermal, intratracheal, epidural, and combinations thereof. The dose of iptakalim hydrochloride is administered in a form of one of a capsule, a cachet, a pill, a tablet, a powder, a granule, a pellet, a bead, a particle, a gum, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, a foam, a food product, and combinations thereof. The method may further comprise the step of administering one of an antihyperglycaemic/antihypertensive agent, an antihyperglycaemic agent, an antihypertensive agent, and combinations thereof. The method may further comprise the step of administering comprises administering a safe and therapeutically effective amount of a combination therapy comprising iptakalim hydrochloride and one of an antihyperglycaemic/antihypertensive agent, an antihyperglycaemic agent, an antihypertensive agent, and combinations thereof.

In still another aspect, this document features a method of treating Type-2 diabetes and one of a cardiovascular disorder, a kidney system disorder, a blood vessel disorder, an eye disorder, and combinations thereof, the method comprising administering to a patient in need thereof a safe and therapeutically effective amount of a composition comprising iptakalim hydrochloride, wherein the amount administered is between about 0.5 milligrams and about 4 milligrams per kilogram of body weight of the patient.

Implementations may include one or more of the following. The amount administered of iptakalim hydrochloride is between about 0.5 milligrams and about 4 milligrams per kilogram of body weight of the patient. The amount administered of iptakalim hydrochloride is about 3 milligrams per kilogram of body weight of the patient. An administration route for the dose of iptakalim hydrochloride is one of topical, buccal, sublingual, transdermal, oral, rectal, ophthalmic, intravitreal, intracameral, nasal, vaginal, parenteral, subcutaneous, intramuscular, intravenous, intradermal, intratracheal, epidural, and combinations thereof. The dose of iptakalim hydrochloride is administered in a form of one of a capsule, a cachet, a pill, a tablet, a powder, a granule, a pellet, a bead, a particle, a gum, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, a foam, a food product, and combinations thereof. The method may further comprise the step of administering one of an antihyperglycaemic/antihypertensive agent, an antihyperglycaemic agent, an antihypertensive agent, and combinations thereof. The method may further comprise the step of administering comprises administering a safe and therapeutically effective amount of a combination therapy comprising iptakalim hydrochloride and one of an antihyperglycaemic/antihypertensive agent, an antihyperglycaemic agent, an antihypertensive agent, and combinations thereof.

In even another aspect, this document features a method for closing ATP-sensitive potassium (KATP) channels comprising administration of iptakalim hydrochloride to pancreatic β-cells in animals, humans and cloned cell lines. Implementations may include one or more of the following. The K_{ATP} channels are SUR1 subunit-containing K_{ATP} channels. The K_{ATP} channels are Kir6.2/SUR1 subunit-containing K_{ATP} channels.

In yet another aspect, this document features a method for increasing insulin release from pancreatic β-cells comprising administration of iptakalim hydrochloride.

These and other aspects and implementations may have one or more of the following advantages. Iptakalim hydrochloride, an established cardiovascular K_{ATP} channel opener, potently blocks β-cell K_{ATP} channels. This unique feature makes iptakalim an optimal antihyperglycaemic agent that exhibits positive benefits in cardiac and blood vessel systems. The development of a new drug that closes pancreatic β-cell K_{ATP} channels but opens cardiovascular K_{ATP} channels has important clinical significances. Large amounts of evidence indicate that the opening of cardiovascular K_{ATP} channels exhibits beneficial effects on cardiovascular disorders, including the protection of the myocardial system against ischemia/hypoxia, the prevention of ventricular arrhythmias and antihypertension. All of these K_{ATP} channel-opening effects will benefit type 2-diabetic patients with accompanying cardiac and blood vessel disorders.

The foregoing and other aspects, features, and advantages will be apparent from the DESCRIPTION and DRAWINGS, and from the CLAIMS.

### DRAWINGS

Implementations will hereinafter be described in conjunction with the appended DRAWINGS, where like designations denote like elements.

FIGS. 1-3 show that like glucose, iptakalim hydrochloride depolarizes and excites pancreatic β-cells.

FIGS. 4-7 show that iptakalim hydrochloride inhibits β-cell K_{ATP} channel-mediated whole-cell currents in a concentration-dependent manner.

FIGS. 8-10 show that iptakalim hydrochloride inhibits β-cell K_{ATP} channel-mediated single currents.

FIGS. 11-14 show that iptakalim hydrochloride induces the decrease of K_{ATP} channel activity independent of intracellular ATP concentration.

FIGS. 15-18 show that iptakalim elevates β-cell intracellular Ca²⁺ concentrations, which is similar to the effects of glucose or the classic sulfonylurea tolbutamide.

FIG. 19 shows that like glucose or tolbutamide, iptakalim significantly increases insulin secretion from rat β-cell islets.

### DESCRIPTION

### A. TERMINOLOGY AND DEFINITIONS

In describing implementations, the following terminology will be used in accordance with the definitions and explanations set out below. Notwithstanding, other terminology, definitions, and explanations may be found throughout this document as well.

As used herein, “treating” refers to amelioration of a disease substantially associated with diabetes mellitus resulting from defects in insulin production, insulin action, or both, which amelioration includes the reduction of detectable blood glucose levels.

As used herein, a disease “substantially associated with” diabetes mellitus means that at least high blood glucose levels are one manifestation of the disease and are present in a significant percentage of diseased patients (e.g., Type-2 diabetes), and/or that accompanying cardiac, kidney system, and blood vessel disorders, such as ischemia/hypoxia, ventricular arrhythmias, antihypertension, and the like, are other manifestations of the disease.

As used herein, “mammal” refers to a member belonging to the class Mammalia. Implementations may be particularly useful in the treatment of human subjects, although they may be intended for veterinary uses as well.

As used herein, a “pharmaceutically acceptable” refers to: 1) materials and compositions that are physiologically tolerable by and suitable for use with mammals, and more particularly with humans, without undue adverse side effects (such as toxicity, irritation, and allergic response) commensurate with a reasonable benefit/risk ratio; and/or 2) approved by a regulatory agency of the Federal or a state government or listed in the U.S. Pharmacopeia or other generally recognized pharmacopeia for use in mammals, and more particularly in humans.

As used herein, a “safe and therapeutically effective amount” refers to the amount of a formulation that may be sufficient to yield a desired therapeutic response without undue adverse side effects (e.g. toxicity, irritation, allergic response) commensurate with a reasonable benefit/risk ratio when used in the manners of this invention. The specific safe and therapeutically effective amount will vary with such factors as the particular condition being treated, the physical condition of the patient, the type of patient being treated, the duration of the treatment, the nature of combination therapy (if any), and the specific formulations employed for example.

As used herein, a “pharmaceutically acceptable salt” refers to a variety of salts of a pharmaceutically acceptable active agent including, for example, salts with inorganic bases, salts with organic bases, salts with inorganic acids, salts with organic acids, salts with basic or acidic amino acids, and the like. Pharmaceutically acceptable salts with inorganic bases are exemplified by alkali metal salts, e.g., sodium salts, potassium salts, and the like; alkaline earth metal salts, e.g., calcium salts, magnesium salts, and the like; as well as aluminum salts, ammonium salts, and the like. Pharmaceutically acceptable salts with organic bases are exemplified by salts of trimethylamine, triethylamine, pyridine, picoline, ethanolamine, diethanolamine, triethanolamine, dicyclohexylamine, N,N′-dibenzylethylenediamine, and the like. Pharmaceutically acceptable salts with inorganic acids are exemplified by those of hydrochloric acid, hydrobromic acid, nitric acid, sulfuric acid, phosphoric acid, and the like. Pharmaceutically acceptable salts with organic acids are exemplified by those of formic acid, acetic acid, trifluoroacetic acid, fumaric acid, oxalic acid, tartaric acid, maleic acid, citric acid, succinic acid, malic acid, methanesulfonic acid, benzensulfonic acid, p-toluenesulfonic acid, and the like. Pharmaceutically acceptable salts with basic amino acids are exemplified by those of arginine, lysine, ornithine, and the like, and the salts with acidic amino acids are those of aspartic acid, glutaric acid, and the like.

As used herein, a “pharmaceutically acceptable carrier” refers to a variety of organic or inorganic carrier materials, including pharmaceutically acceptable excipients, lubricants, binders, disintegrators, diluents, extenders, solvents, suspending agents, dissolution aids, isotonization agents, buffering agents, soothing agents, amphipathic lipid delivery systems, vehicles, and the like materials suitably selected with respect to the intended form of administration and as consistent with conventional pharmaceutical practices for delivering/administering a pharmaceutical composition to a mammal.

As used herein, a “pharmaceutically acceptable additive” refers to a variety of materials, including pharmaceutically acceptable solubilizers, enzyme inhibiting agents, anticoagulants, antifoaming agents, antioxidants, coloring agents, coolants, cryoprotectants, hydrogen bonding agents, flavoring agents, plasticizers, preservatives, sweeteners, thickeners, and the like materials suitably selected with respect to the intended form of administration and as consistent with conventional pharmaceutical practices for facilitating the processes involving the preparation of pharmaceutical compositions.

As used herein, “combination therapy” or “adjunct therapy” means that the patient in need of one pharmaceutically acceptable active agent may be treated with or given another pharmaceutically acceptable active agent or other pharmaceutically acceptable active agents for the disease in conjunction therewith. This combination therapy may be sequential therapy where the patient may be treated first with one pharmaceutically acceptable active agent, and then the other pharmaceutically acceptable active agent or agents are given simultaneously or separately (e.g. within a proximity of hours or days between separate administration of one or more components of the combination).

As used herein, an “antihyperglycaemic/antihypertensive agent” generally refers to any SUR subunit-selective compound that selectively modulates K_{ATP} channels expressed in specific tissues, and more specifically may refer to any compound that blocks directly and/or indirectly pancreatic β-cell K_{ATP} channels while exhibiting little blocking effects on cardiovascular K_{ATP} channels, or even opening cardiovascular K_{ATP} channels (e.g., iptakalim hydrochloride (N-(1-methylethyl)-1,1,2-trimethyl-propylamine hydrochloride)).

As used herein, a “pharmaceutically acceptable active agent” refers to any antihyperglycaemic/antihypertensive agent, any antihyperglycaemic agent, any antihypertensive agent, and/or the like, and/or their physiologically active salts or esters, their combinations with their various salts, their tautomers and/or isomeric forms, their analog forms, their derivative forms, and/or their prodrugs.

### B. COMPOSITIONS

Implementations may provide pharmaceutical compositions for use in treating diseases substantially associated with diabetes mellitus in mammals, such as Type-2 diabetes and/or accompanying cardiac, kidney system, and blood vessel disorders for example.

In an implementation, a pharmaceutical composition may be provided comprising a safe and therapeutically effective amount of a composition comprising an antihyperglycaemic/antihypertensive agent. In another implementation, a pharmaceutical composition may be provided comprising a safe and therapeutically effective amount of a composition comprising an antihyperglycaemic/antihypertensive agent and one of an antihyperglycaemic agent, an antihypertensive agent, and combinations thereof.

Pharmaceutically acceptable active agents suitable for use in the pharmaceutical composition and treatment method implementations are not particularly limited. Such pharmaceutically acceptable active agents may be any substance or mixture of substances having therapeutic or other value when administered to mammals, particularly to a human, for treating diseases substantially associated with diabetes mellitus.

An antihyperglycaemic/antihypertensive agent may be any SUR subunit-selective compound that selectively modulates K_{ATP} channels expressed in specific tissues, and more specifically may be any compound that blocks directly and/or indirectly pancreatic β-cell K_{ATP} channels while exhibiting little blocking effects on cardiovascular K_{ATP} channels, or even opening cardiovascular K_{ATP} channels.

Any of the amine derivatives disclosed in U.S. Patent Application Publication No. 20040266822, which is hereby incorporated entirely herein by reference, pinacidil, the cyanoguaniding group of K_{ATP} channel openers which are based on the structure of pinacidil, and pharmaceutically acceptable salts, esters, tautomers, isomers, analogs, derivatives, prodrugs thereof, and combinations thereof may be some of the possible antihyperglycaemic/antihypertensive agents. For the exemplary purposes of this disclosure, the antihyperglycaemic/antihypertensive agent advantageously used in implementations may be iptakalim hydrochloride (N-(1-methylethyl)-1,1,2-trimethyl-propylamine hydrochloride) and pharmaceutically acceptable salts, esters, tautomers, isomers, analogs, derivatives, and prodrugs thereof, and combinations thereof. Iptakalim hydrochloride has many advantages, including its pharmacological properties (such as being a small molecule, water-soluble, its ability to freely penetrate the blood-brain barrier) and its exhibition of little side effects after long-term systemic administration. The inset in FIG. 2 shows the chemical structure of iptakalim hydrochloride.

Without being bound by theory, it is believed that there may be several possible mechanisms that mediate iptakalim hydrochloride-induced β-cell K_{ATP} channel closure. First, iptakalim hydrochloride may bind to glibenclamide sites of the SUR1 subunit, thereby altering SUR subunit conformation, which in turn may diminish β-cell K_{ATP} channel opening. Second, iptakalim hydrochloride may eliminate β-cell K_{ATP} channel activity by increasing either ATP production or sensitivity of K_{ATP} channels to intracellular ATP. Finally, iptakalim hydrochloride may directly block β-cell K_{ATP} channels by acting on the Kir6.2 subunit.

Glitazones (increase insulin receptor sensitivity) and pharmaceutically acceptable salts, esters, tautomers, isomers, analogs, derivatives, and prodrugs thereof, and combinations thereof may be one of the possible antihyperglycaemic agents. Glitazones increase insulin receptor sensitivity, and when used in combination with iptakalim hydrochloride, may increase its efficacy. Other possible antihyperglycaemic agents that could be utilized in the present invention may be sulfonylureas, including tolbutamide, glyburide, gliclazide, nateglinide, repaglinide and glibenclarimade, and pharmaceutically acceptable salts, esters, tautomers, isomers, analogs, derivatives, and prodrugs thereof, and combinations thereof.

Accordingly, the pharmaceutical composition implementations prepared for the use may contain pharmaceutically acceptable active agents as free substances or in the forms of their physiologically active salts or esters, their combinations with their various salts, their tautomers and/or isomeric forms, their analog forms, their derivative forms, or their prodrugs for example.

Examples of advantageous pharmaceutically acceptable active salts are salts with inorganic bases, salts with organic bases, salts with inorganic acids, salts with organic acids, salts with basic or acidic amino acids, and the like. Pharmaceutically acceptable salts with inorganic bases are exemplified by alkali metal salts, e.g., sodium salts, potassium salts, and the like; alkaline earth metal salts, e.g., calcium salts, magnesium salts (e.g. magnesium citrate), and the like; as well as aluminum salts, ammonium salts, and the like. Pharmaceutically acceptable salts with organic bases are exemplified by salts of diethylamine, trimethylamine, triethylamine, pyridine, picoline, ethanolamine, diethanolamine, triethanolamine, dicyclohexylamine, N,N′-dibenzylethylenediamine, and the like. Pharmaceutically acceptable salts with inorganic acids are exemplified by those of hydrochloric acid, hydrobromic acid, nitric acid, sulfuric acid, phosphoric acid, and the like. Pharmaceutically acceptable salts with organic acids are exemplified by those of formic acid, acetic acid, trifluoroacetic acid, fumaric acid, oxalic acid, tartaric acid, maleic acid, citric acid, succinic acid, malic acid, methanesulfonic acid, benzensulfonic acid, p-toluenesulfonic acid, and the like. Pharmaceutically acceptable salts with basic amino acids are exemplified by those of arginine, lysine, ornithine, and the like, and the salts with acidic amino acids are those of aspartic acid, glutaric acid, and the like.

A prodrug refers to any covalently bonded carrier that releases a pharmaceutically acceptable active agent in vivo when such prodrug is administered to a mammalian subject. Prodrugs may be prepared by modifying functional groups present in the pharmaceutically acceptable active agents in such a way that the modifications are cleaved, either in routine manipulation (e.g. enzymatically oxidized, reduced or hydrolyzed) or in vivo (e.g. hydrolyzed with gastric acid or the like), to the parent pharmaceutically acceptable active agents. Prodrugs may include, for example compounds wherein hydroxy, amine, sulfhydryl, or carboxyl groups are bonded to any group that, when administered to a mammalian subject, cleaves to form a free hydroxyl, amino, sulfhydryl, or carboxyl group, respectively.

Pharmaceutical compositions of the present invention may also include a pharmaceutically acceptable additive (e.g. one of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and combinations thereof) and/or a pharmaceutically acceptable carrier (e.g. one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof) as described in more detail below.

### C. DOSAGE FORMS

Pharmaceutical compositions comprising pharmaceutically acceptable active agents for treating diseases substantially associated with diabetes mellitus are in principle all pharmaceutical administration forms that may be used for any route of administration. Amounts and regimens for the administration of pharmaceutical compositions comprising pharmaceutically acceptable active agents may be determined readily.

Pharmaceutical compositions of the present invention may conveniently be presented in unit dosage form. Unit dosage formulations may be those containing a daily dose or unit, a daily sub-dose, or an appropriate fraction thereof, of the administered ingredients as described herein.

A dosage unit may comprise a pharmaceutically acceptable active agent. In addition, a dosage unit may comprise one or more pharmaceutically acceptable active agents admixed with a pharmaceutically acceptable carrier(s), a pharmaceutically acceptable additive(s), and/or any combination thereof.

Accordingly, the dosage units may be in a form suitable for administration by standard routes. In general, the dosage units may be administered by the topical (including buccal and sublingual), transdermal, oral, rectal, ophthalmic (including intravitreal or intracameral), nasal, vaginal, and/or parenteral (including subcutaneous, intramuscular, intravenous, intradermal, intratracheal, and epidural) routes for example.

For the exemplary purposes of this disclosure, oral delivery may be a particularly advantageous delivery route for administration to mammals of pharmaceutically acceptable active agents, optionally formulated with appropriate pharmaceutically acceptable carriers and pharmaceutically acceptable additives to facilitate application. More particularly and also for the exemplary purposes of this disclosure, oral transmucosal (OT) delivery may be a particularly advantageous delivery route for administration to mammals. One of the advantages of OT delivery is that it is a non-invasive drug delivery method. Furthermore, OT delivery has better patient compliance, less risk of infection and lower cost than invasive procedures such as injection and implantation. It also has much shorter onset time, i.e., the time from administration to therapeutic effect, than does oral delivery. A pharmaceutical composition absorbed via the oral mucosa will also avoid first pass metabolism, in which the drug may be metabolized in the GI tract and liver. OT delivery is simple and is administered by the caregiver or the patient with minimal discomfort.

Pharmaceutically acceptable active agents may be administered alone, but may also be administered in admixture with one or more organic and/or inorganic carrier materials, including pharmaceutically acceptable excipients, lubricants, binders, disintegrators, diluents, extenders, solvents, suspending agents, dissolution aids, isotonization agents, buffering agents, soothing agents, amphipathic lipid delivery systems, soluble polymers, biodegradable polymers, or other like carrier materials (collectively referred to herein as a pharmaceutically acceptable carrier, carrier materials, or carriers), suitably selected with respect to the intended form of administration and as consistent with conventional pharmaceutical practices for delivering/administering a pharmaceutical composition to a mammal. These carriers may be solids or liquids, and the type of carrier may be generally chosen based on the type of administration being used.

Lubricants are any anti-sticking agents, glidants, flow promoters, and the like materials that perform a number of functions in tablet manufacture, for example, such as improving the rate of flow of the tablet granulation, preventing adhesion of the tablet material to the surface of the dies and punches, reducing interparticle friction, and facilitating the ejection of the tablets from the die cavity. Lubricants may include, for example, magnesium stearate, calcium stearate, talc, colloidal silica, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, sodium oleate, sodium stearate, magnesium stearate, sodium chloride, leucine, PEG-4000, magnesium lauryl sulfate, and the like.

Binders are any agents used to impart cohesive qualities to powdered material through particle-particle bonding for example. Binders may include, for example, matrix binders (e.g. dry starch, dry sugars), film binders (e.g. PVP, starch paste, celluloses, bentonite, sucrose), and chemical binders (e.g. polymeric cellulose derivatives, such as methyl cellulose, carboxy methyl cellulose, hydroxy propyl cellulose, hydroxy propyl methyl cellulose); sugar syrups; corn syrup; water soluble polysaccharides such as acacia, tragacanth, guar and alginates; gelatin; gelatin hydrolysate; agar; sucrose; dextrose; non-cellulosic binders, such as polyvinylpyrrolidone (PVP), PEG, vinyl pyrrolidone copolymers, pregelatinized starch, sorbitol, and glucose); and the like.

Disintegrators are any substances that facilitate the breakup or disintegration of tablets after administration for example. Disintegrators may include, for example, starch, starch derivatives, clays (e.g bentonite), algins, gums (e.g. xanthan gum, guar gum), cellulose, cellulose derivatives (e.g methyl cellulose, carboxymethyl cellulose, low-substituted hydroxypropyl cellulose, carboxymethyl cellulose calcium), croscarmellose sodium, carboxymethyl starch sodium, Veegum HV, agar, wood products, natural sponge, ion-exchange resins (e.g. styrene/divinyl benzene copolymers, quaternary ammonium compounds), alginic acid, citrus pulp, cross-linked polyvinylpyrrolidone, sodium starch glycolate, microcrystalline cellulose, and the like.

Diluents are any inert substances added to increase the bulk of the pharmaceutical composition to make the tablet a practical size for compression for example. Diluents may include, for example, calcium phosphate, calcium sulfate, lactose, kaolin, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, dextrose, silica, and the like.

Solvents may include, for example, water, alcohols, ketones, esters, chlorinated hydrocarbons, propylene glycol, macrogol, oils, including those of petroleum, animal, vegetable or synthetic origin, such as peanut oil, soybean oil, mineral oil, sesame oil, corn oil, and the like.

Dissolution aids may include, for example, polyethylene glycol, propylene glycol, D-mannitol, benzyl benzoate, ethanol, trisaminomethane, cholesterol, triethanolamine, sodium carbonate, sodium citrate, and the like.

Suspending agents may include, for example, surface activators such as stearyl triethanolamine, sodium lauryl sulfate, laurylaminopropionic acid, lecithin, benzalkonium chloride, benzethonium chloride, glycerin monostearate, and the like; and hydrophilic high molecular weight materials such as polyvinyl alcohol, polyvinylpyrrolidone, carboxymethylcellulose sodium, methylcellulose, hydroxymethyl cellulose, hydroxyethyl cellulose, hydroxypropyl cellulose, and the like.

Isotonization agents may include, for example, sodium chloride, glycerin, D-mannitol, and the like.

Buffering agents may include, for example: where the acid may be a pharmaceutically acceptable acid, such as hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid; where the base may be a pharmaceutically acceptable base, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine; a salt of a pharmaceutically acceptable cation and acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid; and the like.

Soothing agents may include, for example, benzyl alcohol and the like.

Amphipathic lipid delivery systems, may include, for example, liposomes, such as small unilamellar vesicles, large unilamallar vesicles, multilamellar vesicles, and the like, micelles, and the like. Such lipid delivery systems act as carriers of the active agents that are imbedded in them, protect the active agents during transit through the GI tract for example, and permit a high rate of absorption into the cells. Lipid delivery systems also enhances the biological action in the cells of the active agents.

Pharmaceutically acceptable active agents may also be coupled to soluble polymers as targetable pharmaceutically acceptable active agent carriers. Soluble polymers may include, for example, polyvinylpyrrolidone, pyran copolymer, polyhydroxylpropylmethacrylamide-p-henol, polyhydroxyethylaspartamidephenol, polyethyleneoxide-polylysine substituted with palmitoyl residues, and the like.

Furthermore, pharmaceutically acceptable active agents may be coupled to a class of biodegradable polymers useful in achieving controlled and sustained release of the pharmaceutically acceptable active agents. For example, the polymers may be implanted in the vicinity of where pharmaceutically acceptable active agent delivery may be desired (e.g. at the site of a biofilm forming bacterial infection). Biodegradable polymers may include, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, crosslinked or amphipathic block copolymers of hydrogels, and the like.

The pharmaceutical composition implementations may optionally include one or more pharmaceutically acceptable additives. Suitable additives are those commonly utilized to facilitate the processes involving the preparation of pharmaceutical compositions implementations. Based on the functionality, non-limiting examples of pharmaceutically acceptable additives may include pharmaceutically acceptable solubilizers, enzyme inhibiting agents, anticoagulants, antifoaming agents, antioxidants, coloring agents, coolants, cryoprotectants, hydrogen bonding agents, flavoring agents, plasticizers, preservatives, sweeteners, thickeners, and the like.

Solubilizers are any additives that increase the solubility of the pharmaceutically acceptable active agents and/or other composition components in the pharmaceutically acceptable carrier. Suitable solubilizers for use in the pharmaceutical composition implementations are readily available from standard commercial sources and may include: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide); amides, such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide, and polyvinylpyrrolidone; esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, .delta.-valerolactone and isomers thereof, beta.-butyrolactone and isomers thereof, and other solubilizers known in the art, such as dimethyl acetamide, dimethyl isosorbide (Arlasolve DMI (ICI)), N-methylpyrrolidones (Pharmasolve (ISP)), monooctanoin, diethylene glycol monoethyl ether (available from Gattefosse under the trade name Transcutol), and water.

The amount of any solubilizer that may be included in pharmaceutical compositions of the present invention is not particularly limited. Of course, when such compositions are ultimately administered to a patient, the amount of a given solubilizer may be limited to a bioacceptable amount, which may be readily determined by one of skill in the art. In some circumstances, it may be advantageous to include amounts of solubilizers far in excess of bioacceptable amounts, for example, to maximize the concentration of pharmaceutically acceptable active agents, with excess solubilizer removed prior to providing the composition to a patient using conventional techniques, such as distillation or evaporation.

When a pharmaceutically acceptable active agent is subject to enzymatic degradation, the pharmaceutical compositions may include an enzyme inhibiting agent. Generally, enzyme inhibiting agents may be divided into the following classes: Inhibitors that are not based on amino acids, such as P-aminobenzamidine, FK-448, camostat mesylate, sodium glycocholate; Amino acids and modified amino acids, such as aminoboronic acid derivatives and n-acetylcysteine; Peptides and modified peptides, such as bacitracin, phosphinic acid dipeptide derivatives, pepstatin, antipain, leupeptin, chymostatin, elastatin, bestatin, hosphoramindon, puromycin, cytochalasin potatocarboxy peptidase inhibitor, and amastatin; Polypeptide protease inhibitors, such as aprotinin (bovine pancreatic trypsin inhibitor), Bowman-Birk inhibitor and soybean trypsin inhibitor, chicken egg white trypsin inhibitor, chicken ovoinhibitor, and human pancreatic trypsin inhibitor; Complexing agents, such as EDTA, EGTA, 1,10-phenanthroline and hydroxychinoline; and Mucoadhesive polymers and polymer-inhibitor conjugates, such as polyacrylate derivatives, chitosan, cellulosics, chitosan-EDTA, chitosan-EDTA-antipain, polyacrylic acid-bacitracin, carboxymethyl cellulose-pepstatin, and polyacrylic acid-Bwoman-Birk inhibitor.

The choice and levels of the enzyme inhibitor are based on toxicity, specificity of the proteases, and the potency of the inhibition. The inhibitor may be suspended or solubilized in the pharmaceutical composition preconcentrate, or added to the aqueous diluent.

Without being bound by theory, it is believed that an inhibitor may function solely or in combination as: a competitive inhibitor, by binding at the substrate binding site of the enzyme, thereby preventing the access to the pharmaceutically acceptable active agent, such as antipain, elastatinal and the Bowman Birk inhibitor; a non-competitive inhibitor that is simultaneously bound to the enzyme site along with the pharmaceutically acceptable active agent, as their binding sites are not identical; and/or a complexing agent due to loss in enzymatic activity caused by deprivation of essential metal ions out of the enzyme structure.

Anticoagulants, may include, for example, acetylated monoglycerides and the like.

Antifoaming agents, may include, for example, long-chain alcohols, silicone derivatives, and the like.

Antioxidants, may include, for example, BHT, BHA, gallic acid, propyl gallate, ascorbic acid, ascorbyl palmitate, 4-hydroxymethyl-2,6-di-tert-butyl phenol, tocopherol, sulfites, and the like.

Coloring agents (agents that give tablets a more pleasing appearance, and in addition help the manufacturer to control the product during its preparation and help the user to identify the product) may include, for example, approved certified water-soluble FD&C dyes, lakes (a lake is the combination by adsorption of a water-soluble dye to a hydrous oxide of a heavy metal, resulting in an insoluble form of a dye), natural vegetable colorants, iron oxides, titanium dioxide, silicates, sulfates, magnesium hydroxide, aluminum hydroxide, and the like.

Coolants, may include, for example, halogenated hydrocarbons (e.g., trichloroethane, trichloroethylene, dichloromethane, fluorotrichloromethane), diethylether, liquid nitrogen, and the like.

Cryoprotectants, may include, for example, trehelose, phosphates, citric acid, tartaric acid, gelatin, dextran, mannitol, and the like.

Hydrogen bonding agents, may include, for example, magnesium oxide and the like.

Flavoring agents, may include, for example, esters, alcohols, aldehydes, carbohydrates, complex volatile oils, synthetic flavors, ethyl vanillin, and the like.

Plasticizers, may include, for example, polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol, dibutyl seccate, and the like.

Preservatives, may include, for example, ascorbic acid, boric acid, sorbic acid, benzoic acid, and salts thereof, peroxybenzoic acid esters, dehydroacetic acid, parabens, phenols, chlorobutanol, benzyl alcohol, phenethyl alcohol, quaternary ammonium compounds, and the like.

Sweeteners, may include, for example, natural sweeteners such as stevia, maltose, sucrose, glucose, sorbitol, glycerin and dextrins, artificial sweeteners such as aspartame, saccharine and saccharine salts, and the like.

Thickeners, may include, for example, sugars, polyvinylpyrrolidone, cellulosics, polymers, alginates, and the like.

Pharmaceutically acceptable additives may also be materials such as: proteins (e.g., collagen, gelatin, Zein, gluten, mussel protein, lipoprotein); carbohydrates (e.g., alginates, carrageenan, cellulose derivatives, pectin, starch, chitosan); gums (e.g., xanthan gum, gum arabic); spermaceti; natural or synthetic waxes; carnauba wax; fatty acids (e.g., stearic acid, hydroxystearic acid); fatty alcohols; sugars; shellacs, such as those based on sugars (e.g., lactose, sucrose, dextrose) or starches; polysaccharide-based shellacs (e.g., maltodextrin and maltodextrin derivatives, dextrates, cyclodextrin and cyclodextrin derivatives); cellulosic-based shellacs (e.g., microcrystalline cellulose, sodium carboxymethyl cellulose, hydroxypropylmethyl cellulose, ethyl cellulose, hydroxypropyl cellulose, cellulose acetate, cellulose nitrate, cellulose acetate butyrate, cellulose acetate, trimellitate, carboxymethylethyl cellulose, hydroxypropylmethyl cellulose phthalate); inorganics, such as dicalcium phosphate, hydroxyapitite, tricalcium phosphate, talc and titania; polyols, such as mannitol, xylitol and sorbitol; polyethylene glycol esters; and polymers, such as alginates, poly(lactide coglycolide), gelatin, crosslinked gelatin, and agar-agar.

It should be appreciated that there may be considerable overlap between the above-listed pharmaceutically acceptable carriers and pharmaceutically acceptable additives in common usage, since a given carrier or additive is often classified differently by different practitioners in the field, or is commonly used for any of several different functions. Thus, the foregoing pharmaceutically acceptable carriers and pharmaceutically acceptable additives should be taken as merely exemplary, and not limiting, of the types of carriers and additives that may be included in pharmaceutical composition implementations. The amounts of such carriers and additives may be readily determined according to the particular properties desired.

Pharmaceutically acceptable active agents may be administered orally as discrete units including capsules, cachets, pills, or tablets; as powders, granules, pellets, beads, or particles; as a solution, elixir, syrup, tincture, or suspension in an aqueous liquid or a non-aqueous liquid; or as an oil-in-water liquid emulsion or a water-in-oil emulsion and as a bolus, and the like. They may also be administered parenterally, in sterile liquid dosage forms, or by any other route using known dosage forms.

Tablets are any solid pharmaceutical dosage forms containing a pharmaceutically acceptable active agent or agents to be administered with or without suitable pharmaceutically acceptable carriers and/or additives and prepared either by compression or molding methods well known in the art. Tablets have been in widespread use and remain popular as a dosage form because of the advantages afforded both to the manufacturer (e.g., simplicity and economy of preparation, stability, and convenience in packaging, shipping, and dispensing) and the patient (e.g., accuracy of dosage, compactness, portability, blandness of taste, and ease of administration). Although tablets are most frequently discoid in shape, they may also be round, oval, oblong, cylindrical, or triangular. They may differ greatly in size and weight depending on the amount of the pharmaceutically acceptable active agent or agents present and the intended route of administration. They are divided into two general classes, (1) compressed tablets, and (2) molded tablets.

In addition to the pharmaceutically acceptable active agent or agents, tablets may contain a number pharmaceutically acceptable carriers and/or additives. Pharmaceutically acceptable carriers includes those materials that help to impart satisfactory compression characteristics to the formulation, including diluents, binders, disintegrators, and lubricants. Pharmaceutically acceptable additives include those materials that help to give additional desirable physical characteristics to the finished tablet, such as colors, flavors, and sweetening agents. For instance, for oral administration in the dosage unit form of a tablet or capsule, the pharmaceutically acceptable active agent or agents to be administered may be combined with an oral, non-toxic, pharmaceutically acceptable, inert carrier such as lactose, gelatin, agar, starch, sucrose, glucose methyl cellulose, magnesium stearate, dicalcium phosphate, calcium sulfate, mannitol, sorbitol, and the like. The tablets may be optionally scored so that they may be separated into different dosages.

Tablets and other orally discrete dosage forms, such as capsules, cachets, pills, granules, pellets, beads, and particles, for example, may optionally be coated with one or more enteric coatings, seal coatings, film coatings, barrier coatings, compress coatings, fast disintegrating coatings, or enzyme degradable coatings for example. Multiple coatings may be applied for desired performance. Further, dosage forms may be designed for immediate release, pulsatile release, controlled release, extended release, delayed release, targeted release, synchronized release, or targeted delayed release for example. For release/absorption control, carriers may be made of various component types and levels or thicknesses of coats. Such diverse carriers may be blended in a dosage form to achieve a desired performance. In addition, the dosage form release profile may be effected by a polymeric matrix composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be effected through favorable diffusion, dissolution, erosion, ion-exchange, osmosis or combinations thereof.

Formulations suitable for topical administration in the mouth (e.g., OT delivery) may include lozenges comprising the pharmaceutically acceptable active agent or agents to be administered in a flavored basis, usually sucrose and acacia or tragacanth; pastilles comprising the pharmaceutically acceptable active agent or agents to be administered in an inert basis such as gelatin and glycerin, or sucrose and acacia; and mouthwashes comprising the pharmaceutically acceptable active agent or agents to be administered in a suitable liquid carrier.

For oral administration in liquid dosage form, pharmaceutically acceptable active agent or agents are combined with any oral, non-toxic, pharmaceutically acceptable inert carrier such as ethanol, glycerol, water, and the like. Examples of suitable liquid dosage forms may include solutions or suspensions in water, pharmaceutically acceptable fats and oils, alcohols or other organic solvents, including esters, emulsions, syrups or elixirs, suspensions, solutions, and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Liquid dosage forms for oral administration may also include coloring and flavoring to increase patient acceptance.

Liquid dosage forms for parenteral and/or intravenous administration may include a water soluble pharmaceutically acceptable salt of pharmaceutically acceptable active agent or agents. Parenteral and intravenous forms may also include minerals and other materials to make them compatible with the type of injection or delivery system chosen. Formulations suitable for parenteral administration may include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. Antioxidizing agents such as sodium bisulfite, sodium sulfite, or ascorbic acid, either alone or combined, are suitable stabilizing agents. Also used are citric acid and its salts and sodium EDTA. In addition, parenteral solutions may contain preservatives, such as benzalkonium chloride, methyl- or propyl-paraben, and chlorobutanol. In general, water, a suitable oil, saline, aqueous dextrose (glucose), and related sugar solutions and glycols, such as propylene glycol or polyethylene glycols, may be suitable carriers for parenteral and/or intravenous solutions. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind described herein.

Formulations suitable for topical administration to the skin may be presented as sprays, drops, ointments, creams, gels, pastes, transdermal patches, foams, and combinations thereof comprising the pharmaceutically acceptable active agent or agents to be administered in a pharmaceutically acceptable carrier. To be administered in the form of a transdermal delivery system, the dosage administration may be continuous rather than intermittent throughout the dosage regimen.

Formulations suitable for topical administration to the eyes may be presented as sprays, drops, ointments, creams, gels, pastes, transdermal patches, foams comprising the pharmaceutically acceptable active agent or agents to be administered in a pharmaceutically acceptable carrier. To be administered in the form of a transdermal delivery system, the dosage administration may be continuous rather than intermittent throughout the dosage regimen.

Pharmaceutically acceptable active agent or agents may also be administered in intranasal form via use of suitable intranasal vehicles. Formulations suitable for nasal administration, wherein the carrier is a solid, may include a coarse powder having a particle size, for example, in the range of 20 to 500 microns, which may be administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, may include aqueous or oily solutions of the pharmaceutically acceptable active agent or agents.

Formulations for rectal administration may be presented as a suppository with the pharmaceutically acceptable active agent or agents and a suitable base comprising, for example, cocoa butter or a salicylate.

Formulations suitable for vaginal administration may be presented as pessaries, tamports, creams, gels, pastes, foams or spray formulations containing in addition to the pharmaceutically acceptable active agent or agents such carriers as are known in the art to be appropriate.

Accordingly, for the exemplary purposes of this disclosure, because of the ease in passing through the blood-brain barrier, administration of iptakalim hydrochloride may be accomplished in several ways. Administration of iptakalim hydrochloride is effective when ingested orally, such as through capsules, tablets, powders, liquids, or food products. Iptakalim hydrochloride can also be by integrating it into sprays or lozenges to deliver it sublingually to by-pass liver metabolism. Iptakalim hydrochloride may also be capable of respiratory inhalation. Administration of iptakalim hydrochloride may also be effectively accomplished by preparing iptakalim hydrochloride in injectable forms to be delivered parentally to by-pass liver metabolism and for faster and stronger actions. Iptakalim hydrochloride may be dissolved in injection solution and be prepared either for use as a subcutaneous injection or for use as a direct venous injection or intravenous solution. Iptakalim hydrochloride may also be integrated into a patch so that iptakalim hydrochloride can be administered by dermal application of the patch to the skin. An iptakalim hydrochloride patch can also be prepared with other antidiabetic or antihyperglycaemic agents, such as glitazones for example, for increased efficacy.

Thus, the pharmaceutically acceptable active agent or agents may be mixed with a pharmaceutically acceptable carrier. This carrier may be a solid or liquid and the type may be generally chosen based on the type of administration being used. The pharmaceutically acceptable active agent or agents may be co-administered in the form of a tablet or capsule, an amphipathic lipid delivery system, as an agglomerated powder, or in a liquid form for example. Examples of suitable solid carriers may include lactose, sucrose, gelatin and agar. Capsule or tablets may be easily formulated and may be made easy to swallow or chew; other solid forms may include granules, and bulk powders. Tablets may contain suitable binders, lubricants, diluents, disintegrating agents, coloring agents, flavoring agents, flow-inducing agents, and melting agents. Examples of suitable liquid dosage forms may include solutions or suspensions in water, pharmaceutically acceptable fats and oils, alcohols or other organic solvents, including esters, emulsions, syrups or elixirs, suspensions, solutions, and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavoring and coloring agents. Parenteral and intravenous forms may also include minerals and other materials to make them compatible with the type of injection or delivery system chosen.

### D. DOSAGE

Depending on the particular dosage forms used, the effective dose may be varied. In dosage forms suitable for administration, a pharmaceutically acceptable active agent will ordinarily be present in an amount of about 0.5-95% by weight based on the total weight of the composition. Based on the body weight of the patient, the dosage may be administered in a single daily dose, or the total daily dosage may be administered in divided doses of two, three, or four times daily, or administered in one or more doses from one to three times a week for example. Multiple dosage units may be required to achieve a safe and therapeutically effective amount.

For the exemplary purposes of this disclosure and by way of general guidance, antihyperglycaemic/antihypertensive agents, such as iptakalim hydrochloride for example, may be advantageously administered in a dose of from about 0.5 to about 4.0 mg/kg of body weight, or a dose of about 3 mg/kg of body weight, it being possible to administer a dose in a single dose or in divided doses and to maintain effects for up to 6-9 hours.

A pharmaceutically acceptable active agent may be administered by any conventional means available for use in conjunction with pharmaceuticals, either as an individual pharmaceutically acceptable active agent or in combination with other pharmaceutically acceptable active agents. When an individual pharmaceutically acceptable active agent is administered in combination with other pharmaceutically acceptable active agents, the amount and identity of the pharmaceutically acceptable active agents that may be used in treating diseases substantially associated with diabetes mellitus in mammals, such as Type-2 diabetes and/or accompanying cardiac, kidney system, and blood vessel disorders for example, will vary according to patient response and physiology, type and severity of side effects, the disease being treated, the preferred dosing regimen, patient prognosis or other such factors, and the ratio of the pharmaceutically acceptable active agents will be varied as needed according to the desired therapeutic effect, the observed side-effects of the combination, or other such considerations.

When a pharmaceutically acceptable active agent is administered before or after another pharmaceutically acceptable active agent or agents to treat diseases, the respective doses and the dosing regimen of pharmaceutically acceptable active agents may vary. The combination therapy may be sequential, that is the treatment with one pharmaceutically acceptable active agent first, then the second pharmaceutically acceptable active agent, then the third pharmaceutically acceptable active agent, and so on. The sequential therapy may be within a reasonable time after the completion of the first therapy before beginning the second therapy. For example, treatment with the first pharmaceutically acceptable active agent on day 1, the second pharmaceutically acceptable active agent on day 2, the third pharmaceutically acceptable active agent on day 3, and so on. Alternatively, the combination therapy may be concomitant treatment wherein two or more pharmaceutically acceptable active agents are administered at or substantially at the same time. The concomitant treatment with two or more pharmaceutically acceptable active agents may be in the same daily dose or in separate doses. The exact regimen will depend on the disease being treated, the severity of the disease, and the response to the treatment among other considerations.

For example, a full dosing regimen of one pharmaceutically acceptable active agent may be administered either before or after a full dosing regimen of another pharmaceutically acceptable active agent, or alternating doses of pharmaceutically acceptable active agents may be administered. As a further example, one pharmaceutically acceptable active agent may be administered concomitantly with another pharmaceutically acceptable active agent. As additional examples, pharmaceutically acceptable active agents may be administered hours apart, BID (twice a day), every 1-4 days, or on separate days.

The identity of the specific pharmaceutically acceptable active agent, the pharmaceutically acceptable carriers, the pharmaceutically acceptable additives, and/or the amount of pharmaceutical composition administered will vary widely depending on the species and body weight of mammal and the type of disease being treated among other considerations. The dosage administered will vary depending upon known factors, such as the pharmacodynamic characteristics of a specific active agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect for example.

The pharmaceutically acceptable active agents may be administered together in a single dosage form or separately in two or more different dosage forms. These may be administered independently by the same route or by two or more different routes of administration depending on the dosage forms employed.

Overall, the dose and the range of a pharmaceutically acceptable active agent will depend on the particular active agent and the type of disease being treated.

### E. PROCESSES

The pharmaceutical composition implementations may be prepared by conventional pharmaceutical techniques. Such techniques may include the step of uniformly and intimately bringing into association the pharmaceutically acceptable active agent(s), the pharmaceutically acceptable carrier(s), and/or the pharmaceutically acceptable additive(s), and then, if necessary, shaping the product for example.

It should be appreciated that any of the components of the pharmaceutical composition implementations may be used as supplied commercially, or may be preprocessed by agglomeration, air suspension chilling, air suspension drying, balling, coacervation, comminution, compression, pelletization, cryopelletization, extrusion, granulation, homogenization, inclusion complexation, lyophilization, melting, mixing, molding, pan coating, solvent dehydration, sonication, spheronization, spray chilling, spray congealing, spray drying, or other known processes depending in part on the dosage form desired for example. The various components may also be pre-coated or encapsulated. It will also be clear that appropriate carriers and additives may also be introduced to the composition or during the processes to facilitate the preparation of the dosage forms, depending on the need of the individual process.

### F. METHODS OF TREATMENT

The present invention also provides methods of using pharmaceutical composition implementations. The methods of treatment may be any suitable method that may be effective in the treatment of the particular disease or disorder being treated, such as Type-2 diabetes for example.

Treatment method and combination therapy treatment method implementations may be administered to patients by any means, routes, and/or pharmaceutical compositions that achieve their intended purpose. The patient may be an animal, such as a mammal, and more specifically a human. An administration route may be one of a topical, a buccal, a sublingual, a transdermal, an oral, a rectal, an ophthalmic, a intravitreal, an intracameral, a nasal, a vaginal, a parenteral, a subcutaneous, an intramuscular, an intravenous, an intradermal, an intratracheal, an epidural, and combinations thereof for example.

Treatment method and combination therapy treatment method implementations may also be administered in the form of one of a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, a foam, and combinations thereof.

In one implementation, a method of treating a disease substantially associated with diabetes mellitus, such as Type-2 diabetes and/or accompanying cardiac, kidney system, and blood vessel disorders for example, comprises administering to a patient in need thereof a safe and therapeutically effective amount of a composition comprising iptakalim hydrochloride.

In another implementation, a method of treating a disease substantially associated with diabetes mellitus, such as Type-2 diabetes and/or accompanying cardiac, kidney system, and blood vessel disorders for example, comprises administering to a patient in need thereof a safe and therapeutically effective amount of a composition comprising iptakalim hydrochloride and one of an antihyperglycaemic/antihypertensive agent, an antihyperglycaemic agent, an antihypertensive agent, and combinations thereof.

In still another implementation, a method of treating a disease substantially associated with diabetes mellitus, such as Type-2 diabetes and/or accompanying cardiac, kidney system, and blood vessel disorders for example, comprises administering to a patient in need thereof a safe and therapeutically effective amount of a combination therapy comprising iptakalim hydrochloride and one of an antihyperglycaemic/antihypertensive agent, an antihyperglycaemic agent, an antihypertensive agent, and combinations thereof.

### G. EXAMPLES

The following biological activity experiments further illustrate, not limit, the invention.

1. Overview

Iptakalim hydrochloride is a novel antihypertensive drug and its pharmacological mechanisms include the opening of cardiovascular ATP-sensitive potassium (K_{ATP}) channels. Here, the effects of iptakalim on K_{ATP} channels expressed in rat pancreatic β-cells were examined. Under perforated patch-clamp whole-cell configuration in current-clamp mode, iptakalim depolarized β-cells and induced action potential firing, and under whole-cell patch in voltage-clamp mode, iptakalim reduced ramp pulse-opened K_{ATP} channel currents in a concentration-dependent manner. In both cell-attached and inside-out patch single channel recordings, iptakalim reduced K_{ATP} channel open probability. Florescence imaging (fura-2) demonstrated that iptakalim elevated intracellular Ca²⁺ concentrations, and biochemical measurements illustrated that iptakalim increased insulin release. Collectively, although iptakalim has been shown to serve as a novel K_{ATP} channel opener in both cardiovascular smooth muscle and some central neurons, it appears incapable of opening rat pancreatic β-cell K_{ATP} channels; instead, iptakalim closes these channels. Therefore, iptakalim is a subunit-dependent K_{ATP} channel modulator—it opens cardiovascular K_{ATP} channels but closes pancreatic β-cell K_{ATP} channels. The bipolar regulation of K_{ATP} channels expressed in different tissues by iptakalim provides a new therapeutic strategy for the treatment of type II diabetes without cardiovascular side effects.

Iptakalim was initially designed and synthesized as an antihypertensive drug and exhibited powerful antihypertensive effects in a variety of in vivo and in vitro hypertensive animal models. The molecular mechanisms underlying its antihypertensive effects include the opening of cardiovascular K_{ATP} channels. For example, iptakalim significantly enhanced K⁺ currents under patch-clamp whole-cell recording configuration in smooth muscle cells isolated from pulmonary artery, as well as in isolated rat aorta denuded vascular endothelium. Moreover, it has been confirmed that the specific binding of the K_{ATP} channel opener [³H]P1075 could be displaced by iptakalim in a concentration-dependent manner. These results suggest that iptakalim serves as a K_{ATP} channel opener, thereby opening cardiovascular muscle K_{ATP} channels. In addition, the effects of iptakalim on neuronal K_{ATP} channels have been evaluated. In primary cultured hippocampal neurons, iptakalim selectively enhanced voltage-activated K⁺, but not Na⁺ and Ca²⁺, currents, and the binding of [³H]iptakalim to sulfonylurea (SUR) receptors of K_{ATP} channels in rat cerebral cortex, hippocampus, and striatum was displaced by the K_{ATP} channel openers pinacidil and P1075. Therefore, iptakalim, as a K_{ATP} channel opener in the cardiovascular system, also opens some neuronal K_{ATP} channels. Considering some of its advantages, such as being water-soluble, being able to freely penetrate the blood-brain barrier and its low-toxic side-effects during systemic administration, iptakalim is a compound that serves both as a useful pharmacological tool for the study of K_{ATP} channels and as a therapeutic agent for antihypertension and neuroprotection. However, whether iptakalim affects K_{ATP} channels expressed in pancreatic β-cells and regulates insulin release have been unknown up to now.

K_{ATP} channels belong to a family of inwardly rectifying potassium channel subunits (Kir6.2 or 6.1) each coupled to a SUR binding subunit. K_{ATP} channels are widely expressed in a variety of tissues, including muscle cells, pancreatic β-cells and in various neurons. However, among these tissues SUR subunits have exhibited different levels of expression. K_{ATP} channels expressed in pancreatic β-cells have been extensively studied and their physiological roles in regulation of β-cell excitability and insulin release are well established. It is widely accepted that closure of K_{ATP} channels is a key step in glucose-stimulated insulin secretion, and K_{ATP} channel closers have been applied as classic therapeutic agents for the treatment of type-II diabetes. K_{ATP} channels expressed in pancreatic β-cells are formed by Kir6,2-SUR1, while those expressed in cardiovascular muscle cells are formed by Kir6,2-SUR2A or Kir6,2-SUR2B. It has been postulated that the diversity of SUR subunits determines the pharmacological properties of K_{ATP} channels, and this concept led to the development of SUR subunit-selective compounds that selectively modulate K_{ATP} channels. For example, in the presence of ATP, the K_{ATP} channel opener pinacidil dramatically opened SUR2A-containing (cardiovascular type) K_{ATP} channels, but failed to open SUR1-containing (pancreatic β-cell type) K_{ATP} channels, whereas another K_{ATP} channel opener, diazoxide, dramatically opened SUR1-containing K_{ATP} channels, but exhibited less effects on SUR2A-containing K_{ATP} channels.

Therefore, multiple approaches were employed to examine the effects of iptakalim on pancreatic β-cell K_{ATP} channels, on intracellular Ca²⁺ concentrations and on insulin release.

2. Design and Methods

Acutely dissociated-cultured rat pancreatic β-cells were employed as a cellular model to test iptakalim's pharmacology using patch-clamp recording, florescence Ca²⁺ imaging (fura-2) and biochemical measurements.

Pancreatic β-cell isolation. Isolation of rat islets was performed as previously described. In short, male adult rats (Wistar) were anesthetized with diethyl ether, and 10 ml of Hank's buffered saline (HBSS) containing collagenase (200 U/ml, Wako Chem., Japan) was injected into the common bile duct. The pancreas, swollen with digestion solution, was quickly excised and incubated in a plastic culture bottle for 20 min at 37° C. The suspension obtained by shaking the bottle was filtered through 0.5 mm metal mesh and washed with HBSS, which included 2% bovine serum albumin (BSA). About 100 islets were obtained from one rat using the histopaque (specific gravity 1.077, Sigma, St. Louis, Mo., USA) gradient method. After washing with HBSS, which contained 2% BSA, islets were cultured for 24 h with 5% CO₂ in the tissue culture medium. Separation of islets was carried out using dispase (1000 U/ml, Godo Shusei, Japan) as previously described. Separated cells were again cultured for 1-4 days. Only single cells were chosen for experiments. β-cells were identified by detecting cell responses to 15 mM glucose or 0.5 mM tolbutamide (Sigma, St. Louis, Mo., USA). Patch-clamp recordings. Cultured β-cells were kept in a 35-mm Petri dish, and the dish was placed on the stage of an inverted microscope (IMT-2, Olympus, Tokyo, Japan). Membrane potentials and membrane currents were measured using a patch-clamp amplifier (EPC-7, List Electronic, Darmstadt, Germany). The nystatin-perforation method was used to measure the membrane potential and the standard method was used to measure whole-cell currents. The resistance of the electrode, when filled with the pipette solution, ranged from 2 to 4 MΩ. In order to measure whole-cell membrane current, voltage ramp pulses from −90 to −50 mV were repeatedly applied using a ramp pulse generator (SET-2100, Nihon Kohden, Tokyo, Japan). The membrane capacitance ranged from 8 to 14 pF. Series resistance below 12 MΩ was accepted. Single channel current recordings were carried out by the cell-attached and inside-out configurations. All electrophysiological experiments were carried out at room temperature (22±1° C.). Data of single channel currents were low-pass filtered at 1 KHz, digitized at 10 KHz and analyzed using a single channel current analysis program (Clampfit 9.2, Axon Instruments, Foster City, Calif.). The concentration-inhibition curve created by iptakalim was fitted using Origin 5.0 (Microcal, North Hampton, Mass.).

Fura-2 Ca²⁺ imaging. Isolated islets were placed in a glass-bottom culture dish and then loaded with a HEPES buffer solution (in mM: NaCl 140, KCl 4.7, MgCl₂ 1.2, CaCl₂ 1.0, glucose 10 and HEPES 10) containing 1 μM fura-2/AM (Dojin, Kumamoto, Japan) for 20 min at room temperature. Ca⁺ images were captured using an inverted microscope with 40× Plan-Neofluar objectives (Axiovert 135, Zeiss, Oberkochen, Germany), a silicon intensifier target camera and recorded on a fluorescence-imaging system (Argus 50/CA, Hamamatsu Photonics, Hamamatsu, Japan). Excitation wavelengths were 340 nm and 380 nm, selected from a Xenon light source, and emission wavelength was 510 nm. A change in free intracellular Ca²⁺ concentration ([Ca²⁺]ᵢ) was calculated based on the change in ratio of two fluorescence intensities, F340/F380 (24). All microfluorimetric experiments were carried out at room temperature (22±1° C.).

Measurement of insulin release. Isolated islets were hand-picked under microscopy, and ten islets were distributed to each 35-mm Petri dish with 3 ml of HBSS containing 5.5 mM glucose, 20 mM HEPES, and 2% BSA. After pre-incubation for 60 min, islets were exposed to 100 nM BIM for 60 min, and then stimulated by high glucose (22.5 mM), tolbutamide (0.5 mM) or iptakalim (0.1 mM) for 30 min. Before and after glucose stimulation, some samples were collected for measurement of immuno-reactive insulin (IRI), and stored at −20° C. until the assay. IRI was measured by RIA using anti-human insulin antibody with rat insulin standard (Radio-immunoassay Kit, Insulin “Eiken”, Tokyo Japan).

Solutions and Drugs. The standard external solution contained (in mM): 135 NaCl, 5.6 KCl, 1.2 MgCl₂, 1 CaCl₂, 5 glucose, 10 HEPES and pH 7.3 adjusted with KOH. For perforated patch membrane potential recording, the pipette solution contained (in mM): 100 K-gluconate, 35 KCl, 5 glucose, 0.5 EGTA, 10 HEPES, 200 μg/ml nystatin (Sigma, St. Louis, Mo., USA) and pH 7.2. For conventional whole-cell current recording, the pipette solution contained (in mM): 100 K-gluconate, 35 KCl, 1.2 MgCl₂, 5 glucose, 0.5 EGTA, 10 HEPES and pH 7.2. For cell-attached and inside-out single channel recordings, the pipette solution contained (in mM): 135 KCl, 1.2 MgCl₂, 5 glucose, 0.5 EGTA, 10 HEPES and pH 7.3. The ionic composition of the solution inside the membrane (bath solution) in inside-out recordings was the same as the pipette solution, but the pH of this solution was 7.2. When performing inside-out recordings, ATP was added to the bath solution at various concentrations. The iptakalim hydrochloride (N-(1-methylethyl)-1,1,2-trimethyl-propylamine hydrochloride) was kindly provided by Dr. H. Wang (Institute of Pharmacology and Toxicology, Beijing, China) and diazoxide and tolbutamide were both purchased from Sigma (St. Louis, Mo., USA). β-cells in the experimental bath were continuously exposed to a stream of external solution throughout the experiment.

Statistics. Data are expressed as mean±S.E. of several experiments, and statistical significance was evaluated by the two-tailed paired and unpaired Student's t-tests. p values less than 0.05 were considered to be significant.

3. Results

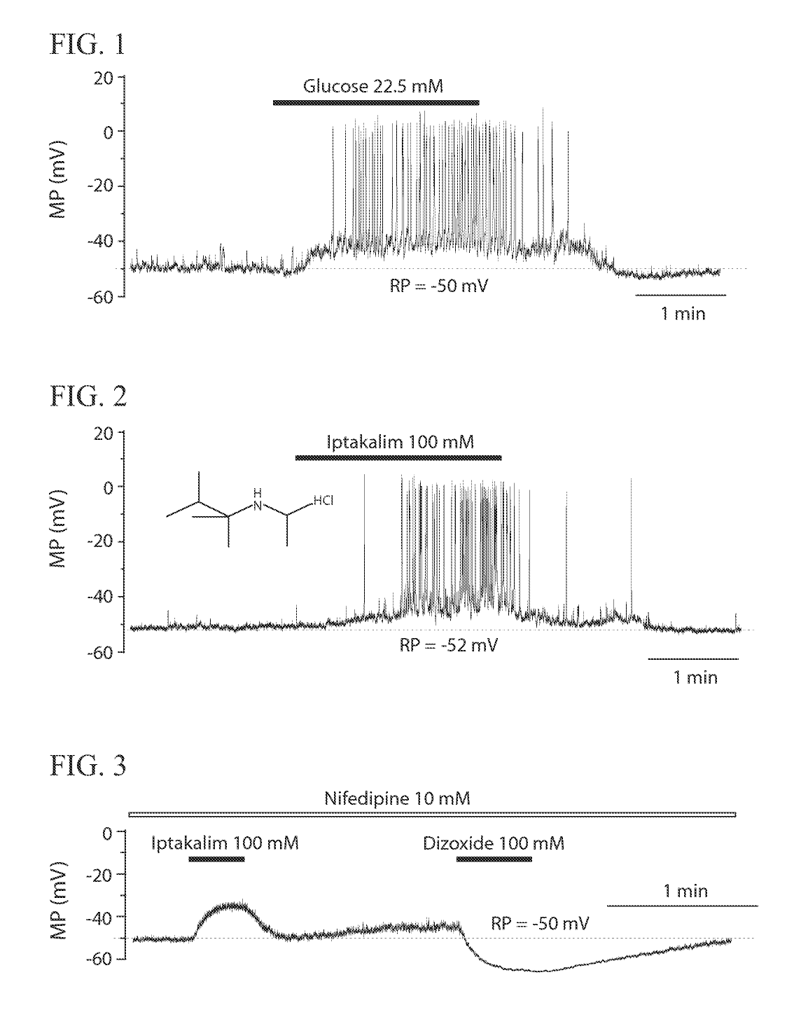

Iptakalim induced electrical excitation of single rat β-cells. Referring to FIGS. 1-3, using nystatin-perforation whole-cell recording in current-clamp mode, the resting membrane potential of rat β-cells was stable (−52.9±1.1 mV, n=28) and the cell was silent (no spontaneous action potential firing) with 5.5 mM glucose in the external solution. Bath-applied 22.5 mM glucose induced action potential firing superimposed on a slow membrane depolarization, which indicated that the high concentration of glucose increased intracellular ATP production, which in turn closed K_{ATP} channels (FIG. 1). With 5.5 mM glucose in the external solution, iptakalim (100 μM) also slowly depolarized the membrane and elicited action potential firing (FIG. 2). In the presence of 10 μM nifedipine (L-type Ca²⁺ channel blocker), which abolished action potential firing, iptakalim (100 μM) clearly depolarized the membrane while a classic K_{ATP} channel opener, diazoxide (100 μM), remarkably hyperpolarized the membrane (FIG. 3). These results suggest that iptakalim regulates pancreatic β-cell excitability by closing K_{ATP} channels. Traces in FIGS. 1-3 are typical cases representative of 6-8 cells tested.

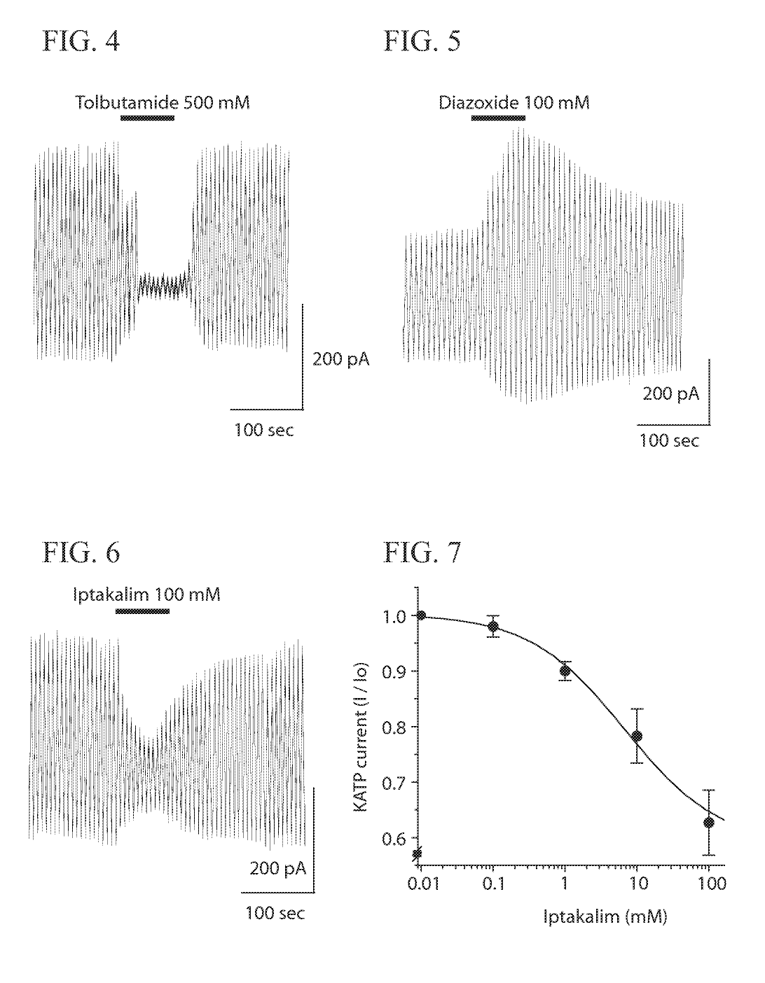

Iptakalim reduced whole-cell current passed through K_{ATP} channels. Turning to FIGS. 4-7, under conventional whole-cell configuration, membrane currents in response to repeatedly-applied voltage ramp pulses from −90 to −50 mV were recorded under voltage-clamp mode (glucose=5.5 mM). The classic K_{ATP} channel blocker tolbutamide (500 μM) reversibly abolished the size of the current (FIG. 4), suggesting that ramp pulse-induced currents were passed through K_{ATP} channels. Bath-application of iptakalim (100 μM) reduced the magnitude of the K_{ATP} channel current (FIG. 6), which was opposite to the observed effects of the same concentration of diazoxide (FIG. 5). FIG. 7 summarizes the concentration-inhibition relationship of iptakalim on β-cell K_{ATP} channels. From seven cells tested, the IC₅₀ and Hill coefficient of iptakalim block of K_{ATP} channel current were 3.3 μM and 0.7, respectively. These results suggest that iptakalim closes β-cell K_{ATP} channels in a concentration-dependent manner. Each symbol was averaged from 6 cells tested and vertical bars represent±SE.

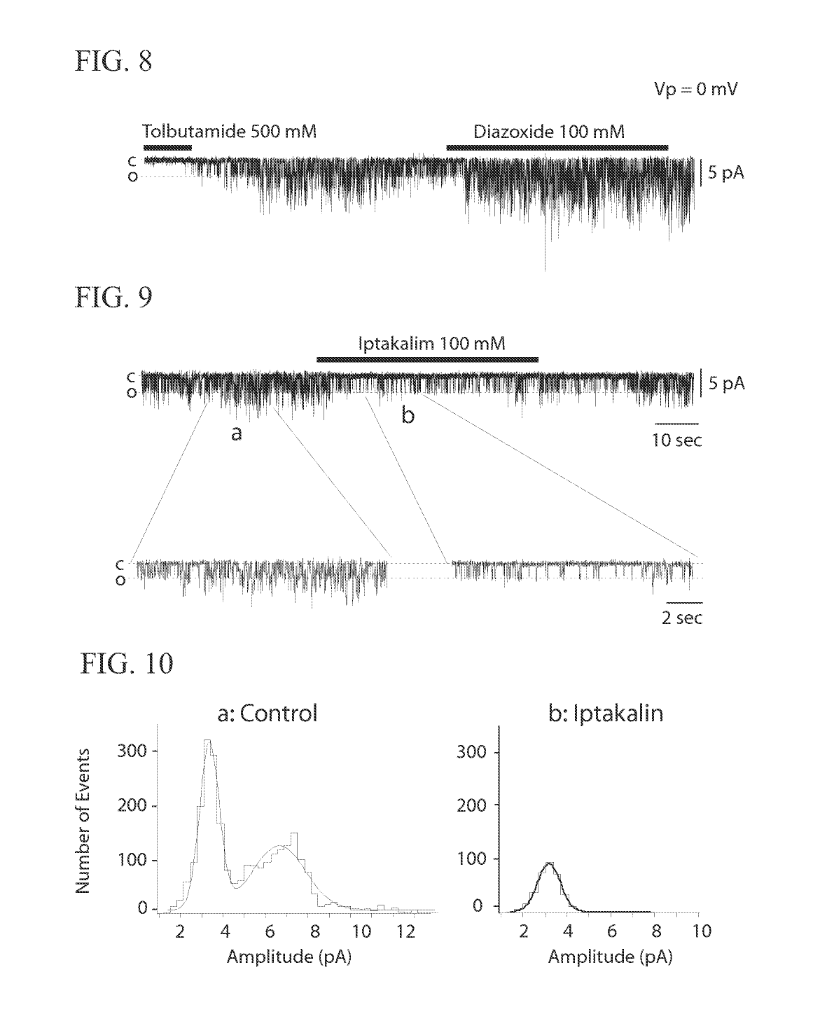

Iptakalim decreased K_{ATP} channel activity in cell-attached single channel recording. Referring to FIGS. 8-10, in cell-attached configuration, spontaneous single channel currents were recorded at a pipette potential (Vp) of 0 mV. Bath-application of tolbutamide (500 μM) completely abolished K_{ATP} channel activity while diazoxide (100 μM) dramatically enhanced K_{ATP} channel activity (FIG. 8), suggesting that the recorded single channel events were passed through K_{ATP} channels. In the same-recorded cell, bath-application of iptakalim (100 μM) reduced channel activities (FIG. 9). The values of open-time probability, mean open time and current amplitude of K_{ATP} channels were 0.43±0.0438, 5.95±0.62 ms and 3.51±0.17 pA before application of iptakalim and 0.121±0.026 (n=17, p<0.001), 8.78±0.81 ms (n=17, p<0.05) and 3.16±0.14 pA (n=17, p<0.01), respectively, during application of iptakalim. FIG. 10 compares K_{ATP} channel current amplitude distribution before and during iptakalim application. The current amplitude distribution histograms show a remarkable decrease of β-cell K_{ATP} channel activity further confirming the antagonist effect of iptakalim on K_{ATP} channels of rat β-cells. c=closed, o=opened.

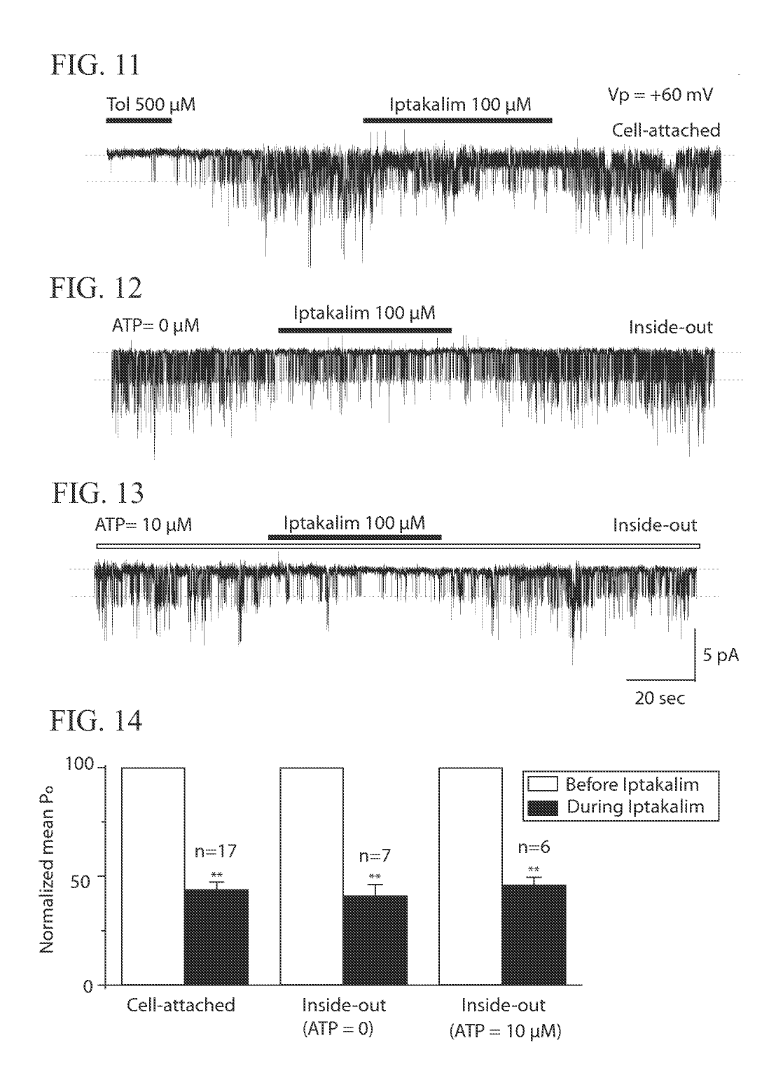

The decrease of K_{ATP} channel activity induced by iptakalim was independent of intracellular ATP concentration. Turning to FIGS. 11-14, in order to examine whether iptakalim blocked β-cell K_{ATP} channels by changing channel sensitivity to ATP, the inside-out recording configuration was employed. As shown in FIG. 11, the cell-attached patch recording demonstrated that spontaneous single channel activities were very sensitive to bath-applied tolbutamide (500 μM) at the Vp of −60 mV, and that 100 μM iptakalim reduced K_{ATP} channel activity. In the inside-out patch recording, application of 100 μM iptakalim reduced K_{ATP} channel activity either in the absence (FIG. 12) or presence (10 μM, FIG. 13) of ATP. The mean open-time probability values were reduced from 0.359±0.065 (n=7) to 0.145±0.041 (n=7, p<001) by 100 μM iptakalim in the absence of ATP, and in the presence of 10 μM ATP, the mean open-time probability values were reduced from 0.222±0.042 (n=7) to 0.103±0.029 (n=6, p<0.05) by 100 μM iptakalim. There were no significant differences of iptakalim-induced inhibition among these recording methods. The tracings in FIGS. 11-13 were recorded from different cells. FIG. 14 compares the inhibitory effects of iptakalim (100 μM) under three single channel recording conditions, and shows similar inhibition in β-cell K_{ATP} channels by iptakalim. These results suggest that iptakalim blocks rat pancreatic β-cell K_{ATP} channels most likely independent of intracellular ATP concentrations.

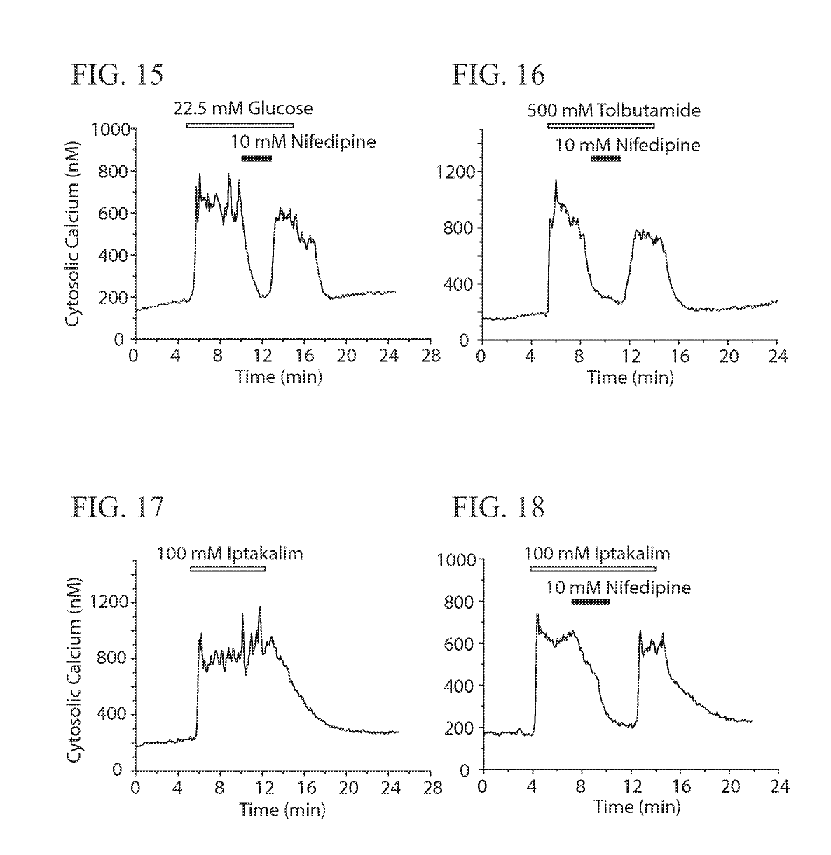

Iptakalim elevated intracellular Ca²⁺ concentrations. Referring to FIGS. 15-18, in order to test whether iptakalim-induced membrane depolarization triggers Ca²⁺ influx through voltage-sensitive Ca²⁺ channels, intracellular Ca²⁺ concentrations were measured using fura-2 florescence imaging. FIG. 15 shows that an increase of glucose concentration from 5.5 to 22.5 mM induced remarkable elevation of intracellular Ca²⁺ concentrations, which was sensitive to the L-type Ca²⁺ channel blocker nifedipine (10 μM, n=6), suggesting that glucose closed K_{ATP} channels, depolarized cell membrane, activated voltage-sensitive Ca²⁺ channels and increased intracellular concentrations. Direct closing of K_{ATP} channels by tolbutamide showed a similar elevation of intracellular Ca²⁺ concentrations through nifedipine-sensitive Ca²⁺ channels (FIG. 16, n=6). Bath-applied 100 μM iptakalim also induced elevation of intracellular Ca²⁺ concentrations (FIG. 17, n=8), which was sensitive to nifedipine as well (FIG. 18, n=5). These results indicate that in rat β-cells, iptakalim closes K_{ATP} channels, depolarizes cell membrane, triggers activation of voltage-sensitive Ca²⁺ channels, and increases intracellular Ca²⁺ concentrations.

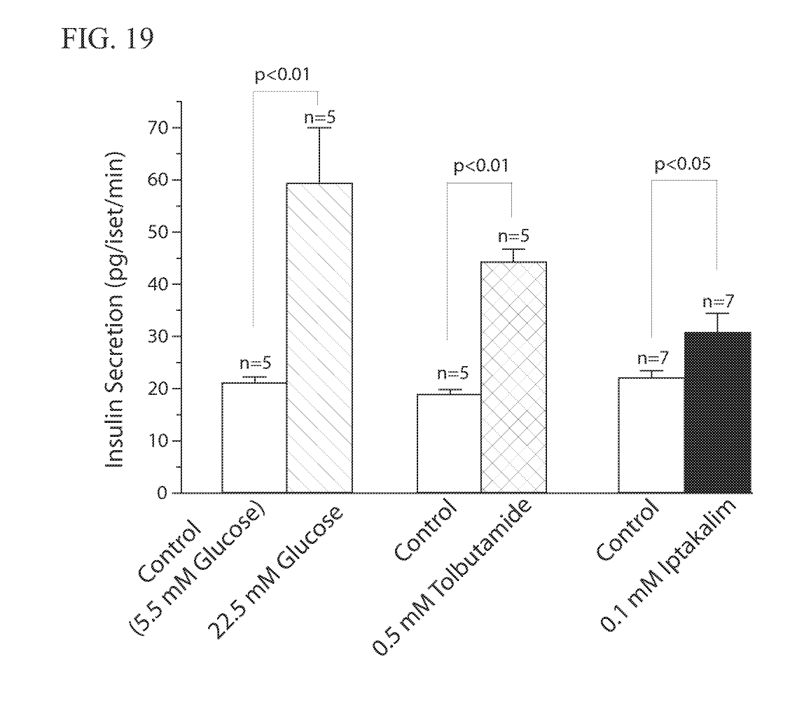

Iptakalim increased insulin release from rat pancreatic islets. Turning finally to FIG. 19, the effects of iptakalim were examined on insulin release. FIG. 19 shows the results of measurement of insulin secretion from rat islets. With glucose at 5.5 mM in the external solution, basal insulin secretion was observed. Application of 22.5 mM glucose increased insulin secretion about 3-fold (p<0.01, n=6), 500 μM tolbutamide increased insulin secretion about 2.5-fold (p<0.01, n=6), and 100 μM iptakalim increased insulin secretion about 1.5-fold (p<0.05, n=8). Each group was averaged from 5-7 cells tested. Vertical bars indicate SE. These data indicate that iptakalim serves as a K_{ATP} channel blocker in rat β-cells, thereby regulating insulin secretion.

4. Discussion

The finding in these studies is that a cardiovascular K_{ATP} channel opener, iptakalim hydrochloride, failed to open rat pancreatic β-cell K_{ATP} channels; instead, iptakalim closed these channels, and in turn, iptakalim excited β-cells, elevated intracellular Ca²⁺ concentrations and increased insulin release. The experimental evidence supporting these findings includes: (1) iptakalim depolarized pancreatic β-cell membrane and elicited action potential firing; (2) iptakalim reduced ramp voltage-induced K_{ATP} currents in a concentration-dependent manner; (3) iptakalim reduced single K_{ATP} channel open probability, which was independent of intracellular ATP concentrations; (4) iptakalim elevated intracellular Ca²⁺ concentrations through nifedipine-sensitive Ca²⁺ channels, and (5) iptakalim increased insulin release. Taken collectively, this is the first report that the cardiovascular K_{ATP} channel opener, iptakalim, inhibits rat pancreatic β-cell K_{ATP} channels. The finding that iptakalim closed β-cell K_{ATP} channels in the present studies, but opened cardiovascular K_{ATP} channels in previous reports provides a new therapeutic strategy for the treatment of patients afflicted with type II diabetes without side effects in the cardiovascular system.

Iptakalim failed to open β-cell K_{ATP} channels. Iptakalim hydrochloride was initially designed as a newly structured K_{ATP} channel opener for antihypertension and exerted remarkable antihypertensive effects in a variety of in vivo and in vitro hypertensive animal models. Accumulating lines of evidence have illustrated that iptakalim directly opens cardiovascular K_{ATP} channels. For instance, using in vitro smooth muscle cells isolated from pulmonary artery, as well as isolated rat aorta denuded vascular endothelium, it has been reported that iptakalim significantly enhanced K⁺ currents using patch-clamp whole-cell recordings. Specific binding of the labeled K_{ATP} channel opener [³H]P1075 was displaced by iptakalim in a concentration-dependent manner. These results indicate that iptakalim opens cardiovascular K_{ATP} channels. In addition, evidence also suggests there is a direct interaction between iptakalim and some neuronal K_{ATP} channels. In primary cultured hippocampal neurons, iptakalim selectively potentiated voltage-activated K⁺, but not Na⁺ and Ca²⁺, channels, and [³H]iptakalim, which was bound to sulfonylurea (SUR) receptors of K_{ATP} channels, could be displaced by the K_{ATP} channel opener pinacidil. In the present study, however, iptakalim failed to open pancreatic β-cell K_{ATP} channels, but the classic β-cell K_{ATP} channel opener diazoxide clearly opened β-cell K_{ATP} channels. Although the precise mechanisms are unclear, the K_{ATP} channel subunits (Kir6.2/SUR1) specially expressed in β-cells may underlie the insensitivity of iptakalim on β-cell K_{ATP} channels.

It is well known that K_{ATP} channels are expressed in a variety of tissues with different SUR subunits. For instance, cardiovascular K_{ATP} channels are formed by Kir6.2-SUR2A, pancreatic β-cell K_{ATP} channels are formed by Kir6.2-SUR1, while midbrain dopamine neurons are formed by Kir6.2-SUR1 (15,25-27) or Kir6.2-SUR2B. This diversity in SUR subunit expression results in different sensibilities to metabolic stress agents and K_{ATP} channel openers. It has been reported that SUR2B exhibited much less susceptibility to the mitochondrial complex I blocker rotenone than SUR1 in substantia nigra neurons. It has also been reported that in the presence of ATP, pinacidil effectively opened K_{ATP} channels co-expressed with Kir6.2-SUR2A, but failed to open K_{ATP} channels co-expressed with Kir6.2-SUR1 in Xenopus oocytes. The sensitivity of K_{ATP} channel openers in two types of central neurons have been compared and it was found that hippocampal CA1 pyramidal neurons were sensitive to the K_{ATP} channel openers diazoxide, pinacidil and lemakalim, whereas A10 DA neurons were only sensitive to diazoxide. This SUR1-subunit specialization of pancreatic β-cells also explains the opening of tolbutamide-sensitive K_{ATP} channels by diazoxide in our experiments since diazoxide was reported to bind to both SUR1 and SUR2 because it opened Kir6.2-SUR1 and Kir6.2-SUR2B channels when MgATP was present. In addition, our current experiments using a transfected Kir6.2/SUR1 cell line (HEK 293) also demonstrated insensitivity to iptakalim but sensitivity to diazoxide (data not shown). Therefore, it is likely that the SUR2A and/or SUR2B, rather than SUR1, subunit of K_{ATP} channels may serve as a sensitive target to mediate iptakalim-induced K_{ATP} channel opening.

Iptakalim closed β-cell K_{ATP} channels. If it is true that iptakalim was not able to open β-cell K_{ATP} channels due to SUR1 subunit expression, the more interesting finding was that iptakalim closed β-cell K_{ATP} channels. Structurally, iptakalim belongs to the cyanoguaniding group of K_{ATP} channel openers which are based on the structure of pinacidil. Thus, in the cardiovascular and nervous systems, iptakalim and pinacidil have exhibited quite similar effects: the opening of K_{ATP} channels. In preliminary experiments, we found that pinacidil also showed inhibition of β-cell K_{ATP} channels (data not shown). Furthermore, it has been reported that PNU-99963, a nonsulphonylurea K_{ATP} channel inhibitor, is also structurally based on pinacidil, making it possible that structurally based K_{ATP} channel opener (especially for pinacidil) analogues (including iptakalim) may also inhibit K_{ATP} channels. Exactly how iptakalim blocks β-cell K_{ATP} channels is unknown, but several possible mechanisms that mediate iptakalim-induced β-cell K_{ATP} channel closure may underlie this inhibition. First, iptakalim may bind to glibenclamide sites of the SUR1 subunit, thereby altering SUR subunit conformation, which in turn may diminish β-cell K_{ATP} channel opening. Emerging evidence has demonstrated that iptakalim-induced pharmacological effects in the cardiovascular and central nervous systems can be prevented by pretreatment with glibenclamide, suggesting that iptakalim and glibenclamide may compete for similar ligand binding sites in SUR2A subunits. In rat pancreatic β-cells, however, this mechanism appears not to be present since β-cell K_{ATP} channels do not express the SUR2A subunit. Second, iptakalim may eliminate β-cell K_{ATP} channel activity by increasing either ATP production or sensitivity of K_{ATP} channels to intracellular ATP. We recently reported that systemic administration of the mitochondrial K_{ATP} channel opener diazoxide protected rats against metabolic stress-induced parkinsonian syndrome in a 6-OHDA-induced Parkinson's disease (PD) model, and that protection by diazoxide was abolished by the relatively selective mitochondrial K_{ATP} channel blocker 5-HD, suggesting that iptakalim may open mitochondrial K_{ATP} channels expressed in rat midbrain cells. Based on these results, it seemed possible that iptakalim would be able to open mitochondrial K_{ATP} channels expressed in rat β-cells, depolarize mitochondrial membrane and alter ATP production. In addition, we previously found that some K_{ATP} channel modulators regulate K_{ATP} channel activity by altering K_{ATP} channel sensitivity to ATP. However, the present results—that iptakalim exhibited quite similar inhibition in the inside-out patch recordings with (10 μM) and without ATP in the bath-do not seem to support these hypotheses. Finally, iptakalim may directly block β-cell K_{ATP} channels by acting on the Kir6.2 subunit. It is well known that some K_{ATP} channel modulators, such as nicorandil, pinacidil or glibenclamide, regulate K_{ATP} channel activity by targeting the regulating subunit SUR, whereas others (e.g., phentolamine and cibenzoline) directly inhibit the pore-forming subunit Kir6.2. Tolbutamide has been shown to act on both SUR1 and Kir6.2. As we have previously discussed, since iptakalim-induced inhibition of β-cell K_{ATP} channels seems to be the result of targeting SUR1, does not increase ATP production and does not alter β-cell K_{ATP} channel sensitivity to ATP, another possible target may be the Kir6.2 subunit. The present single channel analysis demonstrated that the blockade of β-cell K_{ATP} channel activity by iptakalim did not only reduce β-cell K_{ATP} channel mean open-time probability, but also reduced channel current amplitude, suggesting a possible acting site in the Kir6.2 channel pore. However, due to technical limitations in the present study, this hypothesis was not able to be appropriately tested. The heterologously expressed truncated Kir6.2 subunit without the SUR subunit may be the best model to test this hypothesis and such work is currently in progress.

Iptakalim regulated β-cell function and therapeutic implications. In pancreatic β-cells, K_{ATP} channels play a pivotal role in maintaining β-cell membrane potential and regulating β-cell excitation. The closing of these K_{ATP} channels causes β-cell depolarization, in turn activates voltage-sensitive Ca²⁺ channels and increases cytosolic Ca²⁺ concentrations, thereby leading to insulin release. Therefore, the β-cell K_{ATP} channel is a key target for the treatment of type II diabetes mellitus. Indeed, many K_{ATP} channel closers, including tolbutamide, glyburide, gliclazide, nateglinide, repaglinide and glibenclarimade, have been used for many years for the treatment of type II diabetes. On the other hand, K_{ATP} channels are also widely expressed in a variety of other tissues, including the cardiovascular and central nervous systems. Blockade of these K_{ATP} channels due to the treatment of type II diabetes using K_{ATP} channel blockers may cause some severe side effects. For example, it is believed that in the heart, K_{ATP} channels play an important role in the intrinsic mechanisms that protect cardiac muscle during hypoxia/ischemia. In arterial smooth muscle, K_{ATP} channels are also important in maintaining contractile tone, in turn controlling blood pressure and blood flow. In type II diabetic patients treated with K_{ATP} channel blockers, the major cause of death is cardiovascular disease, which has been argued that this could, at least in part, be relevant to the side effects of sulphonylureas by blocking cardiovascular K_{ATP} channels. Therefore, there is a considerable need to develop novel types of pancreatic β-cell K_{ATP} channel blockers which block pancreatic β-cell K_{ATP} channels but exhibit little blocking effects on cardiovascular K_{ATP} channels, or even better, that open cardiovascular K_{ATP} channels. Unfortunately thus far, there are no such optimal reagents that meet these specifications. Although tolbutamide and gliclazide were reported to produce high-affinity closure of β-cell type (Kir6.2/SUR1), but not cardiac type (Kir6.2/SUR2A) or smooth muscle type (Kir6.2/SUR2B) K_{ATP} channels, they exhibit little opening effect on cardiovascular K_{ATP} channels. In the present investigation, it was found, for the first time, that iptakalim closed 1-cell K_{ATP} channels, depolarized β-cells, elevated β-cell intracellular Ca²⁺ concentrations and increased insulin release. The finding that iptakalim, a cardiovascular K_{ATP} channel opener, blocked pancreatic 1-cell K_{ATP} channels indicates that iptakalim is a compound that satisfies therapeutic demands. Evidence has indicated that iptakalim exerts remarkable protective effects against cardiovascular disorders, especially hypertension, in a variety of in vivo and in vitro animal models, and clinical trials for antihypertension are currently being conducted. The unique property of bi-directional regulation of pancreatic β-cells and cardiovascular K_{ATP} channels suggests that iptakalim exhibits great potential to serve as, and stimulate, a new generation of anti-diabetic (type II diabetes) drugs, and it is particularly desirable for the treatment of patients afflicted with type II diabetes accompanied with cardiovascular disorders. Considering its pharmacological properties, such as being a small molecule, water-soluble, its ability to freely penetrate the blood-brain barrier, and because it exhibits little side effects after long-term systemic administration, iptakalim is a promising agent for the treatment of type II diabetes without side effects on, or can even benefit, the cardiovascular system.

Various implementations of the invention are described above. While these descriptions directly describe the above implementations, it is understood that those skilled in the art may conceive modifications and/or variations to the specific implementations shown and described herein. Any such modifications or variations that fall within the purview of this description are intended to be included therein as well. Unless specifically noted, it is the intention of the inventor that words and phrases in the specification and claims be given the ordinary and accustomed meanings to those of ordinary skill in the applicable art(s).

The foregoing DESCRIPTION has been presented and is intended for the purposes of illustration and description. It is not intended to be exhaustive or to limit the invention to the precise form disclosed, and many modifications and variations are possible in the light of the above teachings. The implementations were chosen and described in order to best explain the principles of the invention and its practical application and to enable others skilled in the art to best utilize the invention in various implementations and with various modifications as are suited to the particular use contemplated. Therefore, it is intended that the invention not be limited to the particular implementations disclosed for carrying out this invention, but that the invention will include all implementations falling within the scope of the appended CLAIMS.

1. A method of reducing blood glucose levels in a living organism comprising the step of administering a therapeutically effective dose of iptakalim hydrochloride to the living organism, wherein the amount administered is between about 0.5 milligrams and about 4 milligrams per kilogram of body weight of the living organism.

2. The method of claim 1, wherein the therapeutically effective dose of iptakalim hydrochloride is about 3 milligrams per kilogram of body weight of the living organism.

3. The method of claim 1, wherein an administration route for the dose of iptakalim hydrochloride is one of topical, buccal, sublingual, transdermal, oral, rectal, ophthalmic, intravitreal, intracameral, nasal, vaginal, parenteral, subcutaneous, intramuscular, intravenous, intradermal, intratracheal, epidural, and combinations thereof.

4. The method of claim 1, wherein the dose of iptakalim hydrochloride is administered in a form of one of a capsule, a cachet, a pill, a tablet, a powder, a granule, a pellet, a bead, a particle, a gum, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, a foam, a food product, and combinations thereof.

5. The method of claim 1 further comprising the step of administering one of an antihyperglycaemic/antihypertensive agent, an antihyperglycaemic agent, an antihypertensive agent, and combinations thereof.

6. The method of claim 5, wherein the step of administering comprises administering a therapeutically effective dose of a combination therapy comprising iptakalim hydrochloride and one of an antihyperglycaemic/antihypertensive agent, an antihyperglycaemic agent, an antihypertensive agent, and combinations thereof.

7. The method of claim 1, wherein the living organism is a mammal.

8. The method of claim 7, wherein the mammal is a human.

9. A method of treating Type-2 diabetes comprising administering to a patient in need thereof a safe and therapeutically effective amount of a composition comprising iptakalim hydrochloride, wherein the amount administered is between about 0.5 milligrams and about 4 milligrams per kilogram of body weight of the patient.

10. The method of claim 9, wherein the amount administered of iptakalim hydrochloride is about 3 milligrams per kilogram of body weight of the patient.

11. The method of claim 9, wherein an administration route for the dose of iptakalim hydrochloride is one of topical, buccal, sublingual, transdermal, oral, rectal, ophthalmic, intravitreal, intracameral, nasal, vaginal, parenteral, subcutaneous, intramuscular, intravenous, intradermal, intratracheal, epidural, and combinations thereof.

12. The method of claim 9, wherein the dose of iptakalim hydrochloride is administered in a form of one of a capsule, a cachet, a pill, a tablet, a powder, a granule, a pellet, a bead, a particle, a gum, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, a foam, a food product, and combinations thereof.

13. The method of claim 9 further comprising the step of administering one of an antihyperglycaemic/antihypertensive agent, an antihyperglycaemic agent, an antihypertensive agent, and combinations thereof.

14. The method of claim 13, wherein the step of administering comprises administering a safe and therapeutically effective amount of a combination therapy comprising iptakalim hydrochloride and one of an antihyperglycaemic/antihypertensive agent, an antihyperglycaemic agent, an antihypertensive agent, and combinations thereof.

15. A method of treating Type-2 diabetes and one of a cardiovascular disorder, a kidney system disorder, a blood vessel disorder, an eye disorder, and combinations thereof, the method comprising administering to a patient in need thereof a safe and therapeutically effective amount of a composition comprising iptakalim hydrochloride, wherein the amount administered is between about 0.5 milligrams and about 4 milligrams per kilogram of body weight of the patient.

16. The method of claim 15, wherein the amount administered of iptakalim hydrochloride is about 3 milligrams per kilogram of body weight of the patient.

17. The method of claim 15, wherein an administration route for the dose of iptakalim hydrochloride is one of topical, buccal, sublingual, transdermal, oral, rectal, ophthalmic, intravitreal, intracameral, nasal, vaginal, parenteral, subcutaneous, intramuscular, intravenous, intradermal, intratracheal, epidural, and combinations thereof.

18. The method of claim 15, wherein the dose of iptakalim hydrochloride is administered in a form of one of a capsule, a cachet, a pill, a tablet, a powder, a granule, a pellet, a bead, a particle, a gum, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, a foam, a food product, and combinations thereof.

19. The method of claim 15 further comprising the step of administering one of an antihyperglycaemic/antihypertensive agent, an antihyperglycaemic agent, an antihypertensive agent, and combinations thereof.

20. The method of claim 19, wherein the step of administering comprises administering a safe and therapeutically effective amount of a combination therapy comprising iptakalim hydrochloride and one of an antihyperglycaemic/antihypertensive agent, an antihyperglycaemic agent, an antihypertensive agent, and combinations thereof.
